(19) United States
(12) Patent Application Publication
(10) Pub. No.: US 2004/0058938 A1
(43) Pub. Date: Mar. 25, 2004

(54) Use of substituted imidazoazines, novel imidazoazines, methods for the production thereof, and agents containing these compounds

(76) Inventors: Oliver Cullmann, Heppenheim (DE); Markus Gewehr, Kastellaun (DE); Wassilios Grammenos, Ludwigshafen (DE); Jordi Tormo i Blasco, Limburgerhof (DE); Bernd Muller, Frankenthal (DE); Hubert Sauter, Mannheim (DE); Andreas Gypser, Mannheim (DE); Thomas Grote, Wachenheim (DE); Joachim Rheinheimer, Ludwigshafen (DE); Ingo Rose, Mannheim (DE); Frank Schieweck, Hessheim (DE); Eberhard Ammermann, Heppenheim (DE); Siegfried Strathmann, Limburgerhof (DE); Gisela Lorenz, Hambach (DE); Reinhard Stierl, Mutterstadt (DE)

Correspondence Address: KEIL & WEINKAUF, 1350 CONNECTICUT AVENUE, N.W., WASHINGTON, DC, 20036, US

(21) Appl. No.: 10/433,440

(22) PCT Filed: Dec. 12, 2001
(86) PCT No.: PCT/EP01/14577

(30) Foreign Application Priority Data
Dec. 13, 2000 (DE) 100 61 983.5

(51) Int. Cl. C07D 487/04; C07D 471/04

(52) U.S. Cl. 514/259.1; 514/303; 514/249; 544/282; 544/349; 546/121

(57) ABSTRACT

The use of substituted imidazoazines of the formula I,

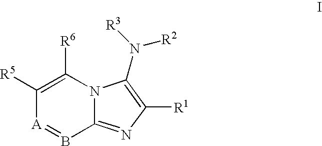

where

R¹ is alkyl, phenyl, phenylalkyl, naphthyl, anthracenyl, cycloalkyl, 5- or 6-membered hetaryl or 5- or 6-membered heterocyclyl, containing one to three nitrogen atoms and/or one oxygen or sulfur atom or one or two oxygen and/or sulfur atoms, or fused 8- to 12-membered hetaryl or fused 8- to 12-membered heterocyclyl, containing one to four nitrogen atoms and/or one oxygen or sulfur atom or one or two oxygen and/or sulfur atoms,

where R¹ may be unsubstituted or substituted as in the description;

R², R³. are hydrogen, alkyl, alkenyl, alkynyl, haloalkyl, haloalkenyl, haloalkynyl, trialkylsilylalkyl, phenyl, phenylalkyl or

R², R³ together with the bridging nitrogen atom form a 5- or 6-membered heterocyclic or heteroaromatic ring which may be interrupted by one to three nitrogen atoms and/or one oxygen or sulfur atom or one or two oxygen and/or sulfur atoms and which may be substituted;

A, B are N or CR⁴;

R⁴, R⁵, R⁶ are hydrogen, halogen, cyano, nitro, hydroxyl, alkyl, haloalkyl, cycloalkyl, alkoxy, haloalkoxy, alkylthio, amino, alkylamino, dialkylamino, alkenyl, alkenyloxy, alkynyl or alkynyloxy;

for controlling phytopathogenic harmful fungi, compositions comprising them, novel imidazoazines and processes for their preparation are described.

The present invention relates to the use of substituted imidazoazines of the formula I,

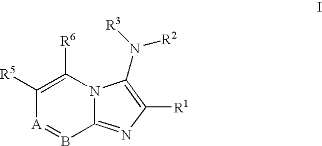

where

R¹ is C₁-C₁₀-alkyl, phenyl, phenyl-C₁-C₄-alkyl, naphthyl, anthracenyl, C₃-C₁₂-cycloalkyl,

where the hydrocarbon radicals are unsubstituted or partially or fully halogenated or may carry one to five identical or different groups R^{a},

R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₃-C₆-alkenyloxy, C₃-C₆-alkynyloxy or C₁-C₄-alkylenedioxy, which may be halogenated,

phenyl, phenoxy, phenyl-C₁-C₄-alkyl, phenyl-C₁-C₄-alkoxy, or

5- or 6-membered hetaryl or 5- or 6-membered heterocyclyl, containing one to three nitrogen atoms and/or one oxygen or sulfur atom or one or two oxygen and/or sulfur atoms, or

fused 8- to 12-membered hetaryl or fused 8- to 12-membered heterocyclyl, containing one to four nitrogen atoms and/or one oxygen or sulfur atom or one or two oxygen and/or sulfur atoms,

where the ring systems are unsubstituted or substituted by one to three radicals R^{b}:

R^{b} is cyano, nitro, amino, aminocarbonyl, aminothiocarbonyl, halogen, hydroxyl, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyloxy, C₂-C₆-alkynyloxy, phenyl, phenoxy, benzyl, benzyloxy, 5- or 6-membered heterocyclyl, 5- or 6-membered hetaryl or 5- or 6-membered hetaryloxy, containing one to three nitrogen atoms and/or one oxygen or sulfur atom or one or two oxygen and/or sulfur atoms,

where the cyclic groups R^{a} and R^{b} for their part are unsubstituted or substituted by one to three groups R^{c}:

R^{c} is cyano, nitro, halogen, hydroxyl, amino, aminocarbonyl, aminothiocarbonyl, C₁-C₆-alkyl, C₁-C₆-haloalkyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy 5- or 6-membered heterocyclyl, 5- or 6-membered hetaryl, 5- or 6-membered hetaryloxy, where the heterocyclic rings are defined as under R^{b};

R², R³ independently of one another are hydrogen, C₁-C₁₀-alkyl, C₃-C_{C}₁₀-alkenyl, C₃-C₁₀-alkynyl, C₁-C_{C}₁₀-haloalkyl, C₃-C₁₀-haloalkenyl, C₃-C₁₀-haloalkynyl, tri(C₁-C₄-alkyl)silyl-C₁-C₄-alkyl, phenyl, phenyl-C₁-C₄-alkyl, or

R², R³ together with the bridging nitrogen atom form a 5- or 6-membered heterocyclic or heteroaromatic ring which may be interrupted by one to three nitrogen atoms and/or one oxygen or sulfur atom or one or two oxygen and/or sulfur atoms and which may be substituted by one or two groups R^{b};

where the cyclic radicals R² and/or R³ may be substituted by one to four groups R^{c};

A, B independently of one another are N or CR⁴;

R⁴, R⁵, R⁶ independently of one another are hydrogen, halogen, cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, amino, C₁-C₆-alkylamino, di-C₁-C₆-alkylamino, C₂-C₆-alkenyl, C₃-C₆-alkenyloxy, C₂-C₆-alkynyl or C₃-C₆-alkynyloxy;

for controlling phytopathogenic harmful fungi, and compositions comprising them.

Moreover, the invention relates to novel imidazoazines and to processes for their preparation.

Substituted imidazoazines having herbicidal activity are known from WO-A 94/18198, EP-A 166 609 and DE-A 41 20 108.

Fungicidally active imidazoazines are known from EP-A 404 190.

In many cases, the activity of the fungicidally active compounds known from the prior art is unsatisfactory.

It is an object of the present invention to provide compounds having improved activity.

We have found that this object is achieved by using substituted imidazoazines of the formula I as fungicide. In addition, we have found novel imidazoazines, compositions comprising them and processes for their preparation.

The compounds of the formula I can be obtained by different synthesis routes.

The imidazoazines of the formula I can be obtained, for example, from halogen derivatives of the formula V by reaction with an amine of the formula VI.

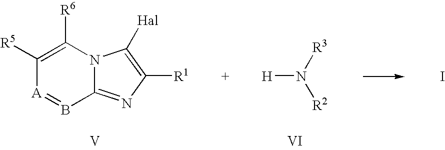

In the formulae V and VI, the variables are as defined for formula I and Hal is halogen, in particular chlorine or bromine. This reaction is usually carried out in an inert solvent with addition of a base and/or a phase-transfer catalyst, at elevated temperature [see WO-A 94/18198 and literature cited therein].

Compounds of the formula I in which the group —NR²R³ is not amino, methylamino or dimethylamino are novel.

Compounds of the formula I in which R³ is hydrogen can be obtained advantageously by the following route:

Amino compounds of the formula II are reacted with aldehydes of the formula III and isonitriles of the formula IV to give imidazoazines of the formula I.

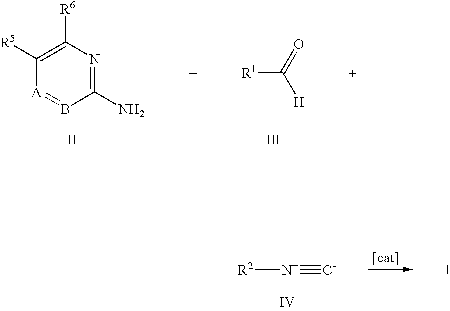

This reaction is usually carried out at temperatures of from −10° C. to 200° C., preferably from 15° C. to 30° C., in an inert organic solvent in the presence of a catalyst [cf. Tetrahedron Lett. 1998, p. 3635].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform-and chlorobenzene, ethers, such as diethyl ether, diisopropyl ether, tert-butyl methyl ether, dioxane, anisole and tetrahydrofuran, nitriles, such as acetonitrile and propionitrile ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethyl formamide and dimethyl acetamide, particularly preferably mixtures of alcohols and halogenated hydrocarbons.

Suitable for use as acids and acidic catalysts are inorganic acids, such as hydrofluoric acid, hydrochloric acid, hydrobromic acid, sulfuric acid and perchloric acid, Lewis acids, such as boron trifluoride, aluminum trichloride, iron(III) chloride, tin(IV) chloride, titanium(IV) chloride and zinc(II) chloride, and also organic acids, such as formic acid, acetic acid, propionic acid, oxalic acid, toluenesulfonic acid, benzenesulfonic acid, camphorsulfonic acid, citric acid and trifluoroacetic acid.

The acids are generally employed in catalytic amounts; however, they can also be used in equimolar amounts, in excess or, if appropriate, as solvent.

Suitable catalysts are, in particular, transition metal salts, preferably from the third transition group. Preference is given to lanthanium(III) compounds and scandium(III) salts. Since the transition metal cation is the catalytically active species, the choice of anion is of minor importance. For practical reasons, preference is given to halides or organic radicals, such as triflate, mesylate or acetate.

The starting materials are generally reacted with one another in equimolar amounts. In terms of yield, it may be advantageous to employ an excess of III and IV, based on II.

The starting materials of the formulae II, III and IV required for preparing the compounds I are commercially available or can be prepared by generally known methods [cf. Houben-Weyl, Methoden der Organischen Synthese [Methods of Organic Chemistry], Vol. E3 (1983) and E5 (1985), Thieme Verlag, Stuttgart and New York].

The isonitriles of the formula IV can be synthesized, for example, from amines of the formula VI. To this end, the amines are reacted by known methods with alkyl formates VII, advantageously under acid catalysis [cf. Heterocycles 1990, p. 1287], to give formamides of the formula VIII which are then dehydrated using POCl₃ with addition of a base such as pyridine, to give isonitriles IV [cf. Org. Syth. Coll. Vol. V, 300].

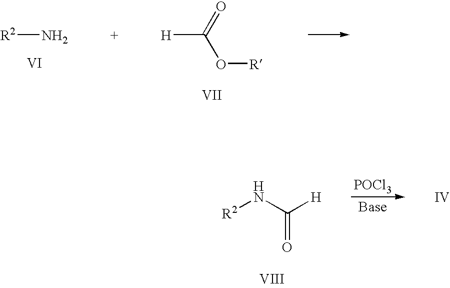

If particular compounds I cannot be obtained by the routes described above, they can be prepared by derivatization of other compounds I.

Intermediates of the formula IV in which R² is (R) CH(CH₃)CF₃, (S) CH(CH₃)CF₃, (R) CH(CH₃)C(CH₃)₃ or (S) CH(CH₃)C(CH₃)₃ are novel.

The reaction mixtures are worked up in a customary manner, for example by mixing with water, phase separation and, if required, chromatographic purification of the crude products. Some intermediates and end products are obtained in the form of colorless or slightly brownish, viscous oils, which are purified or freed from volatile components under reduced pressure and at moderately elevated temperature. If the intermediates and end products are obtained as solids, purification can also be carried out by recrystallization or digestion.

However, if the synthesis yields isomer mixtures, a separation is generally not necessarily required since in some cases the individual isomers can be converted into one another during the preparation for use or upon use (for example under the action of light, acids or bases). Similar conversions may also occur after use, for example in the treatment of plants in the treated plant or in the harmful fungus or animal pest to be controlled.

In the definitions of the symbols in the above formula, collective terms were used which generally represent the following substituents:

halogen: fluorine, chlorine, bromine and iodine;

alkyl: saturated, straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 10 carbon atoms (as mentioned above), it being possible for some or all of the hydrogen atoms in these groups to be replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, 1,1,2,2-tetrafluoroethyl and pentafluoroethyl;

alkenyl: unsaturated, straight-chain or branched hydrocarbon radicals having 2 to 4, 6, 8 or 10 carbon atoms and one double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-2-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated, straight-chain or branched hydrocarbon radicals having 2 to 10 carbon atoms and one double bond in any position (as mentioned above), it being possible for some or all of the hydrogen atoms in these groups to be replaced by halogen atoms as mentioned above, in particular by fluorine, chlorine and bromine;

alkynyl: straight-chain or branched hydrocarbon groups having 2 to 4, 6, 8 or 10 carbon atoms and one triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

haloalkynyl: unsaturated, straight-chain or branched hydrocarbon radicals having 2 to 10 carbon atoms and one triple bond in any position (as mentioned above), it being possible for some or all of the hydrogen atoms in these groups to be replaced by halogen atoms as mentioned above, in particular by fluorine, chlorine and bromine;

cycloalkyl: monocyclic, saturated hydrocarbon groups having 3 to 6, 7, 8, 10 or 12 carbon ring members, for example C₃-C₈-cycloalkyl such as cyclopropyl, cyclobutyl, cyclopentyl, cyclohexyl, cycloheptyl and cyclooctyl;

5- or 6-membered heterocyclyl containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;

fused 8- to 12-membered heterocyclyl, containing one to four nitrogen atoms and/or one oxygen or sulfur atom or one or two oxygen and/or sulfur atoms: saturated or partially unsaturated 5- or 6-membered heterocyclyl groups which, in addition to carbon atoms, may contain one to four nitrogen atoms or one to three nitrogen atoms and one sulfur or oxygen atom as ring members, and in which two adjacent carbon ring members or one nitrogen and one adjacent carbon ring member may be bridged by a buta-1,4-diyl group which may be interrupted by one or two nitrogen atoms;

aryl: a mono- to tricyclic aromatic ring system containing 6 to 14 carbon ring members, for example phenyl, naphthyl and anthracenyl;

5-membered heteroaryl, containing one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring member, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

6-membered heteroaryl, containing one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

fused 8- to 12-membered het roaryl, containing one to four nitrogen atoms and/or one oxygen or sulfur atom or one or two oxygen and/or sulfur atoms: 5- or 6-membered heteroaryl groups which, in addition to carbon atoms, may contain one to four nitrogen atoms or one to three nitrogen atoms and one sulfur or oxygen atom as ring members, and in which two adjacent carbon ring members or one nitrogen and one adjacent carbon ring member may be bridged by a buta-1,3-diene-1,4-diyl group which may be interrupted by one or two nitrogen atoms;

alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;

alkenylene: divalent unbranched chains of 1 to 3 CH₂ groups and one CH═CH group in any position, for example CH═CHCH₂, CH₂CH═CHCH₂, CH═CHCH₂CH₂, CH₂CH═CHCH₂CH₂ and CH═CHCH₂CH₂CH₂.

With respect to the variables, the particularly preferred embodiments of the intermediates correspond to those of the radicals A, B and R¹ to R⁶ of the formula I.

With a view to the intended use of the imidazoazines of the formula I, the following meanings of the substituents are particularly preferred, in each case on their own or in combination:

Particular preference is given to compounds I in which R¹ is phenyl which is substituted by one to five groups R^{b}.

In addition, particular preference is given to compounds of the formula IA:

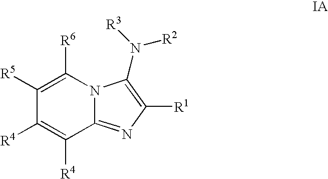

Moreover, particular preference is given to compounds IB:

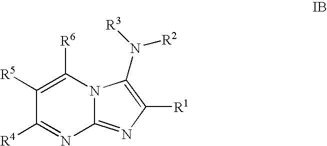

Likewise, particular preference is given to compounds IC:

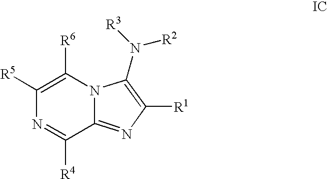

Moreover, particular preference is given to compounds I in which A and B are nitrogen or CH.

Particular preference is given to compounds I in which R¹ is phenyl, benzyl or naphthyl, where these groups may carry one to five substituents selected from the following group: halogen, nitro, cyano, C₁-C₄-alkyl, C₁-C₄-alkoxy, C₁-C₄-alkylthio, C₁-C₄-haloalkyl, C₁-C₄-haloalkoxy, C₁-C₄-haloalkylthio, C₁-C₄-alkylcarbonyl, C₁-C₄-alkoxycarbonyl, di(C₁-C₄-alkyl)amino, phenyl, phenoxy, benzyloxy, where the cyclic substituents may be unsubstituted or substituted by halogen, C₁-C₄-alkyl and C₁-C₄-haloalkyl.

In addition, particular preference is given to compounds I in which R¹ is 5- or 6-membered heteroaryl which is unsubstituted or substituted by up to three radicals R^{b} and which contains one to three nitrogen atoms and/or one oxygen or sulfur atom or one or two oxygen and/or sulfur atoms.

Particular preference is likewise given to compounds I in which R¹ is 6-membered heteroaryl which is unsubstituted or substituted by up to three radicals R^{b} and which contains one to three nitrogen atoms and/or one oxygen or sulfur atom or one or two oxygen and/or sulfur atoms.

In addition, particular preference is given to compounds I in which R¹ is fused heteroaryl which contains one nitrogen atom and is unsubstituted or substituted by up to three radicals R^{b}.

Particular preference is also given to compounds I in which R¹ is phenyl, naphthyl, quinolinyl, isoquinolinyl, pyrimidinyl, pyridinyl, thiophenyl, pyrazolyl, pyrrolyl or furyl, unsubstituted or substituted by up to three radicals R^{b}.

Moreover, particular preference is given to compounds I in which R² is C₂-C₆-alkyl or C₅-C₆-cycloalkyl.

Particular preference is likewise given to compounds I in which R³ is hydrogen.

In addition, particular preference is given to compounds I in which R⁵ and R⁶ are hydrogen.

Furthermore, particular preference is given to compounds I in which R^{b} is halogen, cyano, nitro, C₁-C₄-alkyl, C₁-C₄-haloalkyl, C₁-C₄-alkoxy, C₁-C₄-haloalkoxy, C₁-C₄-alkylamino, di-C₁-C₄-alkylamino or C₁-C₄-alkylthio.

In particular with a view to their use, preference is given to the compounds I compiled in the tables below. Moreover, in their own right, independently of the combination in which they are mentioned, the groups mentioned in the tables for a substituent are a particularly preferred embodiment of the substituent in question.

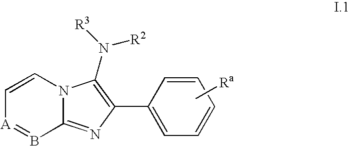

Table 1

Compounds of the formula I.1 in which A and B are CH, R² is hydrogen and R³ is isopropyl and the combination of the radicals R^{a} for a compound corresponds in each case to one row of Table A

Table 2

Compounds of the formula I.1 in which A is N, B is CH, R² is hydrogen and R³ is isopropyl and the combination of the radicals R^{a} for a compound corresponds in each case to one row of Table A

Table 3

Compounds of the formula I.1 in which A is CH, B is N, R² is hydrogen and R³ is isopropyl and the combination of the radicals R^{a} for a compound corresponds in each case to one row of Table A

Table 4

Compounds of the formula I.1 in which A and B are CH, R² is hydrogen and R³ is n-butyl and the combination of the radicals R^{a} for a compound corresponds in each case to one row of Table A

Table 5

Compounds of the formula I.1 in which A is N, B is CH, R² is hydrogen and R³ is n-butyl and the combination of the radicals R^{a} for a compound corresponds in each case to one row of Table A

Table 6

Compounds of the formula I.1 in which A is CH, B is N, R² is hydrogen and R³ is n-butyl and the combination of the radicals R^{a} for a compound corresponds in each case to one row of Table A

Table 7

Compounds of the formula I.1 in which A and B are CH, R² is hydrogen and R³ is isobutyl and the combination of the radicals R^{a} for a compound corresponds in each case to one row of Table A

Table 8

Compounds of the formula I.1 in which A is N, B is CH, R² is hydrogen and R³ is isobutyl and the combination of the radicals R^{a} for a compound corresponds in each case to one row of Table A

Table 9

Compounds of the formula I.1 in which A is CH, B is N, R² is hydrogen and R³ is isobutyl and the combination of the radicals R^{a} for a compound corresponds in each case to one row of Table A

Table 10

Compounds of the formula I.1 in which A and B are CH, R² is hydrogen and R³ is sec-butyl and the combination of the radicals R^{a} for a compound corresponds in each case to one row of Table A

Table 11

Compounds of the formula I.1 in which A is N, B is CH, R² is hydrogen and R³ is sec-butyl and the combination of the radicals R^{a} for a compound corresponds in each case to one row of Table A

Table 12

Compounds of the formula I.1 in which A is CH, B is N, R² is hydrogen and R³ is sec-butyl and the combination of the radicals R^{a} for a compound corresponds in each case to one row of Table A

Table 13

Compounds of the formula I.1 in which A and B are CH, R² is hydrogen and R³ is tert-butyl and the combination of the radicals R^{a} for a compound corresponds in each case to one row of Table A

Table 14

Compounds of the formula I.1 in which A is N, B is CH, R² is hydrogen and R³ is tert-butyl and the combination of the radicals R^{a} for a compound corresponds in each case to one row of Table A

Table 15

Compounds of the formula I.1 in which A is CH, B is N, R² is hydrogen and R³ is tert-butyl and the combination of the radicals R^{a} for a compound corresponds in each case to one row of Table A

Table 16

Compounds of the formula I.1 in which A and B are CH, R² is hydrogen and R³ is cyclopentyl and the combination of the radicals R^{a} for a compound corresponds in each case to one row of Table A

Table 17

Compounds of the formula I.1 in which A is N, B is CH, R² is hydrogen and R³ is cyclopentyl and the combination of the radicals R^{a} for a compound corresponds in each case to one row of Table A

Table 18

Compounds of the formula I.1 in which A is CH, B is N, R² is hydrogen and R³ is cyclopentyl and the combination of the radicals R^{a} for a compound corresponds in each case to one row of Table A

Table 19

Compounds of the formula I.1 in which A and B are CH, R² is hydrogen and R³ is cyclohexyl and the combination of the radicals R^{a} for a compound corresponds in each case to one row of Table A

Table 20

Compounds of the formula I.1 in which A is N, B is CH, R² is hydrogen and R³ is cyclohexyl and the combination of the radicals R^{a} for a compound corresponds in each case to one row of Table A

Table 21

Compounds of the formula I.1 in which A is CH, B is N, R² is hydrogen and R³ is cyclohexyl and the combination of the radicals R^{a} for a compound corresponds in each case to one row of Table A

Table 22

Compounds of the formula I.1 in which A and B are CH, R² is hydrogen and R³ is benzyl and the combination of the radicals R^{a} for a compound corresponds in each case to one row of Table A

Table 23

Compounds of the formula I.1 in which A is N, B is CH, R² is hydrogen and R³ is benzyl and the combination of the radicals R^{a} for a compound corresponds in each case to one row of Table A

Table 24

Compounds of the formula I.1 in which A is CH, B is N, R² is hydrogen and R³ is benzyl and the combination of the radicals R^{a} for a compound corresponds in each case to one row of Table A

Table 25

Compounds of the formula I.1 in which A and B are CH, R² is hydrogen and R³ is trimethylsilylmethyl and the combination of the radicals R^{a} for a compound corresponds in each case to one row of Table A

Table 26

Compounds of the formula I.1 in which A is N, B is CH, R² is hydrogen and R³ is trimethylsilylmethyl and the combination of the radicals R^{a} for a compound corresponds in each case to one row of Table A

Table 27

Compounds of the formula I.1 in which A is CH, B is N, R² is hydrogen and R³ is trimethylsilylmethyl and the combination of the radicals R^{a} for a compound corresponds in each case to one row of Table A

Table 28

Compounds of the formula I.1 in which A and B are CH, R² is hydrogen and R³ is (R)-1,1,1-trifluoroprop-2-yl and the combination of the radicals R^{a} for a compound corresponds in each cast to one row of Table A

Table 29

Compounds of the formula I.1 in which A is N, B is CH, R² is hydrogen and R³ is (R)-1,1,1-trifluoroprop-2-yl and the combination of the radicals R^{a} for a compound corresponds in each case to one row of Table A

Table 30

Compounds of the formula I.1 in which A is CH, B is N, R² is hydrogen and R³ is (R)-1,1,1-trifluoroprop-2-yl and the combination of the radicals R^{a} for a compound corresponds in each case to one row of Table A

Table 31

Compounds of the formula I.1 in which A and B are CH, R² is hydrogen and R³ is (S)-1,1,1-trifluoroprop-2-yl and the combination of the radicals R^{a} for a compound corresponds in each case to one row of Table A

Table 32

Compounds of the formula I.1 in which A is N, B is CH, R² is hydrogen and R³ is (S)-1,1,1-trifluoroprop-2-yl and the combination of the radicals R^{a} for a compound corresponds in each case to one row of Table A

Table 33

Compounds of the formula I.1 in which A is CH, B is N, R² is hydrogen and R³ is (S)-1,1,1-trifluoroprop-2-yl and the combination of the radicals R^{a} for a compound corresponds in each case to one row of Table A

Table 34

Compounds of the formula I.1 in which A and B are CH, R² is hydrogen and R³ is (R)-2,2-dimethylbut-3-yl and the combination of the radicals R^{a} for a compound corresponds in each case to one row of Table A

Table 35

Compounds of the formula I.1 in which A is N, B is CH, R² is hydrogen and R³ is (R)-2,2-dimethylbut-3-yl and the combination of the radicals R^{a} for a compound corresponds in each case to one row of Table A

Table 36

Compounds of the formula I.1 in which A is CH, B is N, R² is hydrogen and R³ is (R)-2,2-dimethylbut-3-yl and the combination of the radicals R^{a} for a compound corresponds in each case to one row of Table A

Table 37

Compounds of the formula I.1 in which A and B are CH, R² is hydrogen and R³ is (S)-2,2-dimethylbut-3-yl and the combination of the radicals R^{a} for a compound corresponds in each case to one row of Table A

Table 38

Compounds of the formula I.1 in which A is N, B is CH, R² is hydrogen and R³ is (S)-2,2-dimethylbut-3-yl and the combination of the radicals R^{a} for a compound corresponds in each case to one row of Table A

Table 39

Compounds of the formula I.1 in which A is CH, B is N, R² is hydrogen and R³ is (S)-2,2-dimethylbut-3-yl and the combination of the radicals R^{a} for a compound corresponds in each case to one row of Table A

Table 40

Compounds of the formula I.1 in which A and B are CH, R² is ethyl and R³ is 2-methylallyl and the combination of the radicals R^{a} for a compound corresponds in each case to one row of Table A

Table 41

Compounds of the formula I.1 in which A is N, B is CH, R² is ethyl and R³ is 2-methylallyl and the combination of the radicals R^{a} for a compound corresponds in each case to one row of Table A

Table 42

Compounds of the formula I.1 in which A is CH, B is N, R² is ethyl and R³ is 2-methylallyl and the combination of the radicals R^{a} for a compound corresponds in each case to one row of Table A

Table 43

Compounds of the formula I.1 in which A and B are CH, R² and R³ together with the bridging nitrogen atom are 4-methylpiperidin-1-yl and the combination of the radicals R^{a} for a compound corresponds in each case to one row of Table A

Table 44

Compounds of the formula I.1 in which A is N, B is CH, R² and R³ together with the bridging nitrogen atom are 4-methylpiperidin-1-yl and the combination of the radicals R^{a} for a compound corresponds in each case to one row of Table A

Table 45

Compounds of the formula I.1 in which A is CH, B is N, R² and R³ together with the bridging nitrogen atom are 4-methylpiperidin-1-yl and the combination of the radicals R^{a} for a compound corresponds in each case to one row of Table A

Table 46

Compounds of the formula I.1 in which A and B are CH, R² and R³ together with the bridging nitrogen atom are morpholin-1-yl and the combination of the radicals R^{a} for a compound corresponds in each case to one row of Table A

Table 47

Compounds of the formula I.1 in which A is N, B is CH, R² and R³ together with the bridging nitrogen atom are morpholin-1-yl and the combination of the radicals R^{a} for a compound corresponds in each case to one row of Table A

Table 48

Compounds of the formula I.1 in which A is CH, B is N, R² and R³ together with the bridging nitrogen atom are morpholin-1-yl and the combination of the radicals R^{a} for a compound corresponds in each case to one row of Table A

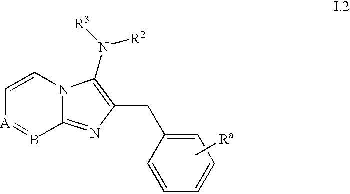

Table 49

Compounds of the formula I.2 in which A and B are CH, R² is hydrogen and R³ is isopropyl and the combination of the radicals, R^{a} for a compound corresponds in each case to one row of Table A

Table 50

Compounds of the formula I.2 in which A is N, B is CH, R² is hydrogen and R³ is isopropyl and the combination of the radicals R^{a} for a compound corresponds in each case to one row of Table A

Table 51

Compounds of the formula I.2 in which A is CH, B is N, R² is hydrogen and R³ is isopropyl and the combination of the radicals R^{a} for a compound corresponds in each case to one row of Table A

Table 52

Compounds of the formula I.2 in which A and B are CH, R² is hydrogen and R³ is n-butyl and the combination of the radicals R^{a} for a compound corresponds in each case to one row of Table A

Table 53

Compounds of the formula I.2 in which A is N, B is CH, R² is hydrogen and R³ is n-butyl and the combination of the radicals R^{a} for a compound corresponds in each case to one row of Table A

Table 54

Compounds of the formula I.2 in which A is CH, B is N, R² is hydrogen and R³ is n-butyl and the combination of the radicals R^{a} for a compound corresponds in each case to one row of Table A

Table 55

Compounds of the formula I.2 in which A and B are CH, R² is hydrogen and R³ is isobutyl and the combination of the radicals R^{a} for a compound corresponds in each case to one row of Table A

Table 56

Compounds of the formula I.2 in which A is N, B is CH, R² is hydrogen and R³ is isobutyl and the combination of the radicals R^{a} for a compound corresponds in each case to one row of Table A

Table 57

Compounds of the formula I.2 in which A is CH, B is N, R² is hydrogen and R³ is isobutyl and the combination of the radicals R^{a} for a compound corresponds in each case to one row of Table A

Table 58

Compounds of the formula I.2 in which A and B are CH, R² is hydrogen and R³ is sec-butyl and the combination of the radicals R^{a} for a compound corresponds in each case to one row of Table A

Table 59

Compounds of the formula I.2 in which A is N, B is CH, R² is hydrogen and R³ is sec-butyl and the combination of the radicals R^{a} for a compound corresponds in each case to one row of Table A

Table 60

Compounds of the formula I.2 in which A is CH, B is N, R² is hydrogen and R³ is sec-butyl and the combination of the radicals R^{a} for a compound corresponds in each case to one row of Table A

Table 61

Compounds of the formula I.2 in which A and B are CH, R² is hydrogen and R³ is tert-butyl and the combination of the radicals R^{a} for a compound corresponds in each case to one row of Table A

Table 62

Compounds of the formula I.2 in which A is N, B is CH, R² is hydrogen and R³ is tert-butyl and the combination of the radicals R^{a} for a compound corresponds in each case to one row of Table A

Table 63

Compounds of the formula I.2 in which A is CH, B is N, R² is hydrogen and R³ is tert-butyl and the combination of the radicals R^{a} for a compound corresponds in each case to one row-of Table A

Table 64

Compounds of the formula I.2 in which A and B are CH, R² is hydrogen and R³ is cyclopentyl and the combination of the radicals R^{a} for a compound corresponds in each case to one row of Table A

Table 65

Compounds of the formula I.2 in which A is N, B is CH, R² is hydrogen and R³ is cyclopentyl and the combination of the radicals R^{a} for a compound corresponds in each case to one row of Table A

Table 66

Compounds of the formula I.2 in which A is CH, B is N, R² is hydrogen and R³ is cyclopentyl and the combination of the radicals R^{a} for a compound corresponds in each case to one row of Table A

Table 67

Compounds of the formula I.2 in which A and B are CH, R² is hydrogen and R³ is cyclohexyl and the combination of the radicals R^{a} for a compound corresponds in each case to one row of Table A

Table 68

Compounds of the formula I.2 in which A is N, B is CH, R² is hydrogen and R³ is cyclohexyl and the combination of the radicals R^{a} for a compound corresponds in each case to one row of Table A

Table 69

Compounds of the formula I.2 in which A is CH, B is N, R² is hydrogen and R³ is cyclohexyl and the combination of the radicals R^{a} for a compound corresponds in each case to one row of Table A

Table 70

Compounds of the formula I.2 in which A and B are CH, R² is hydrogen and R³ is benzyl and the combination of the radicals R^{a} for a compound corresponds in each case to one row of Table A

Table 71

Compounds of the formula I.2 in which A is N, B is CH, R² is hydrogen and R³ is benzyl and the combination-of the radicals R^{a} for a compound corresponds in each case to one row of Table A

Table 72

Compounds of the formula I.2 in which A is CH, B is N, R2 is hydrogen and R³ is benzyl and the combination of the radicals R^{a} for a compound corresponds in each case to one row of Table A

Table 73

Compounds of the formula I.2 in which A and B are CH, R² is hydrogen and R³ is trimethylsilylmethyl and the combination of the radicals R^{a} for a compound corresponds in each case to one row of Table A

Table 74

Compounds of the formula I.2 in which A is N, B is CH, R² is hydrogen and R³ is trimethylsilylmethyl and the combination of the radicals R^{a} for a compound corresponds in each case to one row of Table A

Table 75

Compounds of the formula I.2 in which A is CH, B is N, R² is hydrogen and R³ is trimethylsilylmethyl and the combination of the radicals R^{a} for a compound corresponds in each case to one row of Table A

Table 76

Compounds of the formula I.2 in which A and B are CH, R² is hydrogen and R³ is (R)-1,1,1-trifluoroprop-2-yl and the combination of the radicals R^{a} for a compound corresponds in each case to one row of Table A

Table 77

Compounds of the formula I.2 in which A is N, B is CH, R² is hydrogen and R³ is (R)-1,1,1-trifluoroprop-2-yl and the combination of the radicals R^{a} for a compound corresponds in each case to one row of Table A

Table 78

Compounds of the formula I.2 in which A is CH, B is N, R² is hydrogen and R³ is (R)-1,1,1-trifluoroprop-2-yl and the combination of the radicals R^{a} for a compound corresponds in each case to one row of Table A

Table 79

Compounds of the formula I.2 in which A and B are CH, R² is hydrogen and R³ is (S)-1,1,1-trifluoroprop-2-yl and the combination of the radicals R^{a} for a compound corresponds in each case to one row of Table A

Table 80

Compounds of the formula I.2 in which A is N, B is CH, R² is hydrogen and R³ is (S)-1,1,1-trifluoroprop-2-yl and the combination of the radicals R^{a} for a compound corresponds in each case to one row of Table A

Table 81

Compounds of the formula I.2 in which A is CH, B is N, R² is hydrogen and R³ is (S)-1,1,1-trifluoroprop-2-yl and the combination of the radicals R^{a} for a compound corresponds in each case to one row of Table A

Table 82

Compounds of the formula I.2 in which A and B are CH, R² is hydrogen and R³ is (R)-2,2-dimethylbut-3-yl and the combination of the radicals R^{a} for a compound corresponds in each case to one row of Table A

Table 83

Compounds of the formula I.2 in which A is N, B is CH, R² is hydrogen and R³ is (R)-2,2-dimethylbut-3-yl and the combination of the radicals R^{a} for a compound corresponds in each case to one row of Table A

Table 84

Compounds of the formula I.2 in which A is CH, B is N, R² is hydrogen and R³ is (R)-2,2-dimethylbut-3-yl and the combination of the radicals R^{a} for a compound corresponds in each case to one row of Table A

Table 85

Compounds of the formula I.2 in which A and B are CH, R² is hydrogen and R³ is (S)-2,2-dimethylbut-3-yl and the combination of the radicals R^{a} for a compound corresponds in each case to one row of Table A

Table 86

Compounds of the formula I.2 in which A is N, B is CH, R² is hydrogen and R³ is (S)-2,2-dimethylbut-3-yl and the combination of the radicals R^{a} for a compound corresponds in each case to one row of Table A

Table 87

Compounds of the formula I.2 in which A is CH, B is N, R² is hydrogen and R³ is (S)-2,2-dimethylbut-3-yl and the combination of the radicals R^{a} for a compound corresponds in each case to one row of Table A

Table 88

Compounds of the formula I.2 in which A and B are CH, R² is ethyl and R³ is 2-methylallyl and the combination of the radicals R^{a} for a compound corresponds in each case to one row of Table A

Table 89

Compounds of the formula I.2 in which A is N, B is CH; R² is ethyl and R³ is 2-methylallyl and the combination of the radicals R^{a} for a compound corresponds in each case to one row of Table A

Table 90

Compounds of the formula I.2 in which A is CH, B is N, R² is ethyl and R³ is 2-methylallyl and the combination of the radicals R^{a} for a compound corresponds in each case to one row of Table A

Table 91

Compounds of the formula I.2 in which A and B are CH, R² and R³ together with the bridging nitrogen atom are 4-methylpiperidin-1-yl and the combination of the radicals R^{a} for a compound corresponds in each case to one row of Table A

Table 92

Compounds of the formula I.2 in which A is N, B is CH, R² and R³ together with the bridging nitrogen atom are 4-methylpiperidin-1-yl and the combination of the radicals R^{a} for a compound corresponds in each case to one row of Table A

Table 93

Compounds of the formula I.2 in which A is CH, B is N, R² and R³ together with the bridging nitrogen atom are 4-methylpiperidin-1-yl and the combination of the radicals R^{a} for a compound corresponds in each case to one row of Table A

TABLE A

| No. | R^{a} |
|---|---|
| A-1 | H |
| A-2 | 2-F |
| A-3 | 3-F |
| A-4 | 4-F |
| A-5 | 2-Cl |
| A-6 | 4-Cl |
| A-7 | 2-Br |
| A-8 | 3-Br |
| A-9 | 4-Br |
| A-10 | 2-I |
| A-11 | 3-I |
| A-12 | 4-I |
| A-13 | 2-NO₂ |
| A-14 | 3-NO₂ |
| A-15 | 4-NO₂ |
| A-16 | 2-CN |
| A-17 | 3-CN |
| A-18 | 4-CN |
| A-19 | 2-CH₃ |
| A-20 | 3-CH₃ |
| A-21 | 4-CH₃ |
| A-22 | 2-CF₃ |
| A-23 | 3-CF₃ |
| A-24 | 4-CF₃ |
| A-25 | 2-OCH₃ |
| A-26 | 3-OCH₃ |
| A-27 | 2-SCH₃ |
| A-28 | 3-SCH₃ |
| A-29 | 4-SCH₃ |
| A-30 | 2-OCF₃ |
| A-31 | 3-OCF₃ |
| A-32 | 4-OCF₃ |
| A-33 | 2-OCHF₂ |
| A-34 | 3-OCHF₂ |
| A-35 | 4-OCHF₂ |
| A-36 | 2-SCF₃ |
| A-37 | 3-SCF₃ |
| A-38 | 4-SCF₃ |
| A-39 | 2-OCF₂CHF₂ |
| A-40 | 3-OCF₂CHF₂ |
| A-41 | 4-OCF₂CHF₂ |
| A-42 | 2-COCH₃ |
| A-43 | 3-COCH₃ |
| A-44 | 4-COCH₃ |
| A-45 | 2-COOCH₃ |
| A-46 | 3-COOCH₃ |
| A-47 | 4-COOCH₃ |
| A-48 | 2-C₆H₅ |
| A-49 | 3-C₆H₅ |
| A-50 | 4-C₆H₅ |
| A-51 | 2-(4-F—C₆H₄) |
| A-52 | 3-(4-F—C₆H₄) |
| A-53 | 4-(4-F—C₆H₄) |
| A-54 | 2-(4-CF₃—C₆H₄) |
| A-55 | 3-(4-CF₃—C₆H₄) |
| A-56 | 4-(4-CF₃—C₆H₄) |
| A-57 | 2-O—C₆H₅ |
| A-58 | 3-O—C₆H₅ |
| A-59 | 2-OCH₂—C₆H₅ |
| A-60 | 3-OCH₂—C₆H₅ |
| A-61 | 4-OCH₂—C₆H₅ |
| A-62 | 2-O-(4-Cl—C₆H₄) |
| A-63 | 3-O-(4-Cl—C₆H₄) |
| A-64 | 4-O-(4-Cl—C₆H₄) |
| A-65 | 2-O-(4-CH₃—C₆H₄) |
| A-66 | 3-O-(4-CH₃—C₆H₄) |
| A-67 | 4-O-(4-CH₃—C₆H₄) |
| A-68 | 2-O-[4-C(CH₃)₃—C₆H₄] |
| A-69 | 3-O-[4-C(CH₃)₃—C₆H₄] |
| A-70 | 4-O-[4-C(CH₃)₃—C₆H₄] |
| A-71 | 2-O-(3-CF₃—C₆H₄) |
| A-72 | 3-O-(3-CF₃—C₆H₄) |
| A-73 | 4-O-(3-CF₃—C₆H₄) |
| A-74 | 2-O-(3,4-Cl₂—C₆H₃) |
| A-75 | 3-O-(3,4-Cl₂—C₆H₃) |
| A-76 | 4-O-(3,4-Cl₂—C₆H₃) |
| A-77 | 2-O-(3,5-Cl₂—C₆H₃) |
| A-78 | 3-O-(3,5-Cl₂—C₆H₃) |
| A-79 | 4-O-(3,5-Cl₂—C₆H₃) |
| A-80 | 2,3-F₂ |
| A-81 | 2,4-F₂ |
| A-82 | 3,4-F₂ |
| A-83 | 2,5-F₂ |
| A-84 | 3,5-F₂ |
| A-85 | 2,4-Cl₂ |
| A-86 | 2,5-Cl₂ |
| A-87 | 2,6-Cl₂ |
| A-88 | 2,3-Cl₂ |
| A-89 | 3,4-Cl₂ |
| A-90 | 3,4-Br₂ |
| A-91 | 3,5-Br₂ |
| A-92 | 2,3-(CH₃)₂ |
| A-93 | 2,4-(CH₃)₂ |
| A-94 | 2,5-(CH₃)₂ |
| A-95 | 2,6-(CH₃)₂ |
| A-96 | 3,4-(CH₃)₂ |
| A-97 | 3,5-(CH₃)₂ |
| A-98 | 3,5-(CF₃)₂ |
| A-99 | 2,3-(OCH₃)₂ |
| A-100 | 3,4-(OCH₃)₂ |
| A-101 | 3,5-(OCH₃)₂ |
| A-102 | 2,5-(OCH₃)₂ |
| A-103 | 2-Cl,6-F |
| A-104 | 3-Cl,4-F |
| A-105 | 3-F,4-Cl |
| A-106 | 2-F,3-Cl |
| A-107 | 3-Br,4-F |
| A-108 | 5-Br,2-F |
| A-109 | 2-Br,4-F |
| A-110 | 2-Br,5-F |
| A-111 | 3-NO₂,4-Cl |
| A-112 | 2-NO₂,5-Cl |
| A-113 | 2,3-O—CH₂—O— |
| A-114 | 3-NO₂,4-CH₃ |
| A-115 | 2-F,5-NO₂ |
| A-116 | 3-CN,4-F |
| A-117 | 2-F,5-CH₃ |
| A-118 | 2-CH₃,3-F |
| A-119 | 3-CH₃,4-F |
| A-120 | 4-CH₃,3-F |
| A-121 | 2-F,3-CF₃ |
| A-122 | 2-F,5-CF₃ |
| A-123 | 4-F,3-CF₃ |
| A-124 | 3-F,4-CF₃ |
| A-125 | 5-F,2-CF₃ |
| A-126 | 2-Cl,5-CF₃ |
| A-127 | 2-Cl,3-CF₃ |
| A-128 | 3-CF₃,4-Cl |
| A-129 | 2-F,5-OCH₃ |
| A-130 | 3-F,4-OCH₃ |
| A-131 | 4-F,3-OCH₃ |
| A-132 | 5-F,2-OCH₃ |
| A-133 | 3-Cl,4-OCH₃ |
| A-134 | 5-Cl,2-OCH₃ |
| A-135 | 3-Br,4-OCH₃ |
| A-136 | 2-OCH₃,5-Br |
| A-137 | 3-Cl,4-OCF₃ |
| A-138 | 3-OC₆H₅,4-F |
| A-139 | 3-CH₃,4-N(CH₃)₂ |
| A-140 | 3-OCH₂C₆H₅,4-CH₃ |
| A-141 | 3-CH₃,4-OCH₂—C₆H₅ |
| A-142 | 3-OCH₃,4-OCH₂—C₆H₅ |
| A-143 | 2,3,4-F₃ |
| A-144 | 2,3,5-F₃ |
| A-145 | 2,3,6-F₃ |
| A-146 | 2,4,5-F₃ |
| A-147 | 2,4,6-F₃ |
| A-148 | 3,4,5-F₃ |
| A-149 | 2,3,5-Cl₃ |
| A-150 | 2,3,6-Cl₃ |
| A-151 | 2,4,5-(CH₃)₃ |
| A-152 | 2,3,4-(OCH₃)₃ |
| A-153 | 2,4,5-(OCH₃)₃ |
| A-154 | 2,4,6-(OCH₃)₃ |
| A-155 | 3,4,5-(OCH₃)₃ |
| A-156 | 2,6-F₂,3-Cl |
| A-157 | 2,4-Cl₂,5-F |
| A-158 | 2-Cl,3,6-F₂ |
| A-159 | 2,6-F₂,3-CH₃ |
| A-160 | 2,3-F₂,3-CF₃ |
| A-161 | 3,4-F₂,5-CF₃ |
| A-162 | 2,6-F₂,4-OCH₃ |
| A-163 | 3,5-(OCH₃)₂,4-Br |
| A-164 | 2,3-F₂,4-CH₃ |
| A-165 | 3-Br,4,5-(OCH₃)₂ |
| A-166 | 2-Cl,3-CH₃,6-F |
| A-167 | 2-F,3-CH₃,6-Cl |
| A-168 | 2,3-(CH₃)₂,4-OCH₃ |
| A-169 | 2,5-(CH₃)₂,4-OCH₃ |
| A-170 | 2-CH₃,4,5-(OCH₃)₂ |
| A-171 | 2,3,5,6-F₄ |
| A-172 | F₅ |
| A-173 | (CH₃)₅ |

Table 94

Compounds of the formula I in which A and B are CH, R¹ is 2,6-difluorophenyl, R⁵ and R⁶ are hydrogen and the combination of the radicals R² and R³ for a compound corresponds in each case to one row of Table B

Table 95

Compounds of the formula I in which A is N, B is CH, R¹ is 2,6-difluorophenyl, R⁵ and R⁶ are hydrogen and the combination of the radicals R² and R³ for a compound corresponds in each case to one row of Table B

Table 96

Compounds of the formula I in which A is CH, B is N, R¹ is 2,6-difluorophenyl, R⁵ and R⁶ are hydrogen and the combination of the radicals R² and R³ for a compound corresponds in each case to one row of Table B

Table 97

Compounds of the formula I in which A and B are CH, R¹ is 3-chlorophenyl, R⁵ and R⁶ are hydrogen and the combination of the radicals R² and R³ for a compound corresponds in each case to one row of Table B

Table 98

Compounds of the formula I in which A is N, B is CH, R¹ is 3-chlorophenyl, R⁵ and R⁶ are hydrogen and the combination of the radicals R² and R³ for a compound corresponds in each case to one row of Table B

Table 99

Compounds of the formula I in which A is CH, B is N, R¹ is 3-chlorophenyl, R⁵ and R⁶ are hydrogen and the combination of the radicals R² and R³ for a compound corresponds in each case to one row of Table B

Table 100

Compounds of the formula I in which A and B are CH, R¹ is 3-phenoxyphenyl, R⁵ and R⁶ are hydrogen and the combination of the radicals R² and R³ for a compound corresponds in each case to one row of Table B

Table 101

Compounds of the formula I in which A is N, B is CH, R¹ is 3-phenoxyphenyl, R⁵ and R⁶ are hydrogen and the combination of the radicals R² and R³ for a compound corresponds in each case to one row of Table B

Table 102

Compounds of the formula I in which A is CH, B is N, R¹ is 3-phenoxyphenyl, R⁵ and R⁶ are hydrogen and the combination of the radicals R² and R³ for a compound corresponds in each case to one row of Table B

Table 103

Compounds of the formula I in which A and B are CH, R¹ is 4-methoxyphenyl, R⁵ and R⁶ are hydrogen and the combination of the radicals R² and R³ for a compound corresponds in each case to one row of Table B

Table 104

Compounds of the formula I in which A is N, B is CH, R¹ is 4-methoxyphenyl, R⁵ and R⁶ are hydrogen and the combination of the radicals R² and R³ for a compound corresponds in each case to one row of Table B

Table 105

Compounds of the formula I in which A is CH, B is N, R¹ is 4-methoxyphenyl, R⁵ and R⁶ are hydrogen and the combination of the radicals R² and R³ for a compound corresponds in each case to one row of Table B

Table 106

Compounds of the formula I in which A and B are CH, R¹ is 2,3-dimethylphenyl, R⁵ and R⁶ are hydrogen and the combination of the radicals R² and R³ for a compound corresponds in each case to one row of Table B

Table 107

Compounds of the formula I in which A is N, B is CH, R¹ is 2,3-dimethylphenyl, R⁵ and R⁶ are hydrogen and the combination of the radicals R² and R³ for a compound corresponds in each case to one row of Table B

Table 108

Compounds of the formula I in which A is CH, B is N, R¹ is 2,3-dimethylphenyl, R⁵ and R⁶ are hydrogen and the combination of the radicals R² and R³ for a compound corresponds in each case to one row of Table B

Table 109

Compounds of the formula I in which A and B are CH, R¹ is naphth-1-yl, R⁵ and R⁶ are hydrogen and the combination of the radicals R² and R³ for a compound corresponds in each case to one row of Table B

Table 110

Compounds of the formula I in which A is N, B is CH, R¹ is naphth-1-yl, R⁵ and R⁶ are hydrogen and the combination of the radicals R² and R³ for a compound corresponds in each case to one row of Table B

Table 111

Compounds of the formula I in which A is CH, B is N, R¹ is naphth-1-yl, R⁵ and R⁶ are hydrogen and the combination of the radicals R² and R³ for a compound corresponds in each case to one row of Table B

Table 112

Compounds of the formula I in which A and B are CH, R¹ is naphth-2-yl, R⁵ and R⁶ are hydrogen and the combination of the radicals R² and R³ for a compound corresponds in each case to one row of Table B

Table 113

Compounds of the formula I in which A is N, B is CH, R¹ is naphth-2-yl, R⁵ and R⁶ are hydrogen and the combination of the radicals R² and R³ for a compound corresponds in each case to one row of Table B

Table 114

Compounds of the formula I in which A is CH, B is N, R¹ is naphth-2-yl, R⁵ and R⁶ are hydrogen and the combination of the radicals R² and R³ for a compound corresponds in each case to one row of Table B

Table 115

Compounds of the formula I in which A and B are CH, R¹ is 3,5-dichlorophenyl, R⁵ and R⁶ are hydrogen and the combination of the radicals R² and R³ for a compound corresponds in each case to one row of Table B

Table 116

Compounds of the formula I in which A is N, B is CH, R¹ is 3,5-dichlorophenyl, R⁵ and R⁶ are hydrogen and the combination of the radicals R² and R³ for a compound corresponds in each case to one row of Table B

Table 117

Compounds of the formula I in which A is CH, B is N, R¹ is 3,5-dichlorophenyl, R⁵ and R⁶ are hydrogen and the combination of the radicals R² and R³ for a compound corresponds in each case to one row of Table B

TABLE B

| No. | R² | R³ |
|---|---|---|
| B-1 | H | CH(CH₃)₂ |
| B-2 | CH₃ | CH(CH₃)₂ |
| B-3 | CH₂CH₃ | CH(CH₃)₂ |
| B-4 | CH₂CH₂CH₃ | CH(CH₃)₂ |
| B-5 | H | (CH₂)₃CH₃ |
| B-6 | H | CH₂CH(CH₃)₂ |
| B-7 | H | CH(CH₃)CH₂CH₃ |
| B-8 | H | C(CH₃)₃ |
| B-9 | H | cyclo-C₅H₉ |
| B-10 | CH₃ | cyclo-C₅H₉ |
| B-11 | CH₂CH₃ | cyclo-C₅H₉ |
| B-12 | CH₂CH₂CH₃ | cyclo-C₅H₉ |
| B-13 | H | cyclo-C₆H₁₁ |
| B-14 | H | C₆H₅ |
| B-15 | CH₃ | C₆H₅ |
| B-16 | CH₂CH₃ | C₆H₅ |
| B-17 | CH₂CH₂CH₃ | C₆H₅ |
| B-18 | H | CH₂C₆H₅ |
| B-19 | CH₃ | CH₂C₆H₅ |
| B-20 | CH₂CH₃ | CH₂C₆H₅ |
| B-21 | CH₂CH₂CH₃ | CH₂C₆H₅ |
| B-22 | H | CH₂CH₂C₆H₅ |
| B-23 | CH₃ | CH₂CH₂C₆H₅ |
| B-24 | CH₂CH₃ | CH₂CH₂C₆H₅ |
| B-25 | CH₂CH₂CH₃ | CH₂CH₂C₆H₅ |
| B-26 | H | CH₂Si(CH₃)₃ |
| B-27 | H | (R) CH(CH₃)CF₃ |
| B-28 | H | (S) CH(CH₃)CF₃ |
| B-29 | H | (R) CH(CH₃)C(CH₃)₃ |
| B-30 | H | (S) CH(CH₃)C(CH₃)₃ |
| B-31 | H | CH₂C(═CH₂)CH₃ |
| B-32 | CH₃ | CH₂C(═CH₂)CH₃ |
| B-33 | CH₂CH₃ | CH₂C(═CH₂)CH₃ |
| B-34 | CH₂CH₂CH₃ | CH₂C(═CH₂)CH₃ |
| B-35 | H | CH₂C≡CH |
| B-36 | CH₃ | CH₂C≡CH |
| B-37 | CH₂CH₃ | CH₂C≡CH |
| B-38 | CH₂CH₂CH₃ | CH₂C≡CH |

| | |
|---|---|
| B-39 | —(CH₂)₂CH(CH₃) (CH₂)₂— |
| B-40 | —(CH₂)₂O(CH₂)₂— |

The compounds I are suitable as fungicides. They have outstanding activity against a broad spectrum of phytopathogenic fungi, in particular from the classes of the Ascomycetes, Deuteromiycetes, Phycomycetes and Basidiomiycetes. Some of them act systemically, and they can be employed in crop protection as foliar- and soil-acting fungicides.

They are especially important for controlling a large number of fungi on a variety of crop plants such as wheat, rye, barley, oats, rice, maize, grass, bananas, cotton, soya, coffee, sugar cane, grapevines, fruit species, ornamentals and vegetables such as cucumbers, beans, tomatoes, potatoes and cucurbits, and on the seeds of these plants.

Specifically, they are suitable for controlling the following plant diseases:

Alternaria species, Podosphaera species, Sclerotinia species, Physalospora canker on vegetables and fruit,

Botrytis cinerea (gray mold) on strawberries, vegetables, ornamentals and grapevines,

Corynespora cassiicola on cucumbers,

Colletotrichum species on fruit and vegetables,

Diplocarpon rosae on roses,

Elsinoe fawcetti and diaporthe citri on citrus fruits,

Sphaerotheca species on cucurbits, strawberries and roses,

Cercospora species on groundnuts, sugar beet and aubergines,

Erysiphe cichoracearum on cucurbits,

Leveillula taurica on peppers, tomatoes and aubergines,

Mycosphaerella species on apples and Japanese apricots,

Phyllactinia kakicola, Gloesporium kaki on Japanese apricots,

Gymnosporangium yamadae, Leptothyrium pomi, Podosphaera leucotricha and Gloedes pomigena on apples,

Cladosporium carpophilum on pears and Japanese apricots,

Phomopsis species on pears,

Phytophthora species on citrus fruits, potatoes, onions, in particular Phytophthora infestans on potatoes and tomatoes,

Blumeria graminis (powdery mildew) on cereals,

Fusarium and Verticillium species on various plants,

Glomerella cingulata on tea,

Drechslera and Bipolaris species on cereals and rice,

Mycosphaerella species on bananas and groundnuts,

Plasmopara viticola on grapevines,

Personospora species on onions, spinach and chrysanthemums,

Phaeoisariopsis vitis and Sphaceloma ampelina on grapefruits,

Pseudocercosporella herpotrichoides on wheat and barley,

Pseudoperonospora species on hops and cucumbers,

Puccinia species and Typhula species on cereals and lawn,

Pyricularia oryzae on rice,

Rhizoctonia species on cotton, rice and lawn,

Stagonospora nodorum and Septoria tritici on wheat,

Uncinula necator on grapevines,

Ustilago species on cereals and sugar cane, and

Venturia species (scab) on apples and pears.

Moreover, the compounds I are suitable for controlling harmful fungi such as Paecilomyces variotii in the protection of materials (e.g. wood, paper, paint dispersions, fibers and tissues) and in the protection of stored products.

The compounds I are applied by treating the fungi or the plants, seeds, materials or the soil to be protected against fungal infection, with a fungicidally active amount of the active compounds. Application can be effected both before and after infection of the materials, plants or seeds by the fungi.

In general, the fungicidal compositions comprise from 0.1 to 95, preferably 0.5 to 90, % by weight of active compound.

When used in crop protection, the rates of application are from 0.01 to 2.0 kg of active compound per ha, depending on the nature of the effect desired.

In the treatment of seed, amounts of active compound of from 0.001 to 0.1 g, preferably 0.01 to 0.05 g, are generally required per kilogram of seed.

When used in the protection of materials or stored products, the rate of application of active compound depends on the nature of the field of application and on the effect desired. Rates of application conventionally used in the protection of materials are, for example, from 0.001 g to 2 kg, preferably 0.005 g to 1 kg, of active compound per cubic meter of material treated.

The compounds I can be converted into the customary formulations, e.g. solutions, emulsions, suspensions, dusts, powders, pastes and granules. The use form depends on the particular purpose; in any case, it should ensure a fine and uniform distribution of the compound according to the invention.

The formulations are prepared in a known manner, e.g. by extending the active compound with solvents and/or carriers, if desired using emulsifiers and dispersants, it also being possible to use other organic solvents as auxiliary solvents if water is used as the diluent. Auxiliaries which are suitable are essentially: solvents such as aromatics (e.g. xylene), chlorinated aromatics (e.g. chlorobenzenes), paraffins (e.g. mineral oil fractions), alcohols (e.g. methanol, butanol), ketones (e.g. cyclohexanone), amines (e.g. ethanolamine, dimethylformamide) and water; carriers such as ground natural minerals (e.g. kaolins, clays, talc, chalk) and ground synthetic minerals (e.g. highly-disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid dibutylnaphthalenesulfonic acid, alkylarylsulfonates, alkyl sulfates, alkylsulfonates, fatty alcohol sulfates and fatty acids and their alkali metal and alkaline earth metal salts, salts of sulfated fatty alcohol glycol ether, condensates of sulfonated naphthalene and naphthalene derivatives with formaldehyde, condensates of naphthalene or of naphthalenesulfonic acid with phenol or formaldehyde, polyoxyethylene octylphenol ether, ethoxylated isooctylphenol, octylphenol, nonylphenol, alkylphenol polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, e.g. benzene, toluene, xylene, paraffin, tetrahydronaphthalene, alkylated naphthalenes or their derivatives, methanol, ethanol, propanol, butanol, chloroform, carbon tetrachloride, cyclohexanol, cyclohexanone, chlorobenzene, isophorone, strongly polar solvents, e.g. dimethylformamide, dimethyl sulfoxide, N-methylpyrrolidone and water.

Powders, materials for scattering and dusts can be prepared by mixing or concomitantly grinding the active substances with a solid carrier.

Granules, e.g. coated granules, impregnated granules and homogeneous granules, can be prepared by binding the active compounds to solid carriers. Examples of solid carriers are mineral earths, such as silicas, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, e.g. ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight; of the active compound. The active compounds are employed in a purity of from 90% to 100%, preferably 95% to 100% (according to NMR spectrum).

The following are exemplary formulations:

I. 5 parts by weight of a compound according to the invention are mixed intimately with 95 parts by weight of finely divided kaolin. This gives a dust which comprises 5% by weight of the active compound.

II. 30 parts by weight of a compound according to the invention are mixed intimately with a mixture of 92 parts by weight of pulverulent silica gel and 8 parts by weight of paraffin oil which had been sprayed onto the surface of this silica gel. This gives a formulation of the active compound with good adhesion properties (comprises 23% by weight of active compound).

III. 10 parts by weight of a compound according to the invention are dissolved in a mixture composed of 90 parts by weight of xylene, 6 parts by weight of the adduct of 8 to 10 mol of ethylene oxide and 1 mol of oleic acid N-monoethanolamide, 2 parts by weight of calcium dodecylbenzenesulfonate and 2 parts by weight of the adduct of 40 mol of ethylene oxide and 1 mol of castor oil (comprises 9% by weight of active compound).

IV. 20 parts by weight of a compound according to the invention are dissolved in a mixture composed of 60 parts by weight of cyclohexanone, 30 parts by weight of isobutanol, 5 parts by weight of the adduct of 7 mol of ethylene oxide and 1 mol of isooctylphenol and 5 parts by weight of the adduct of 40 mol of ethylene oxide and 1 mol of castor oil (comprises 16% by weight of active compound).

V. 80 parts by weight of a compound according to the invention are mixed thoroughly with 3 parts by weight of sodium diisobutylnaphthalene-alpha-sulfonate, 10 parts by weight of the sodium salt of a lignosulfonic acid from a sulfite waste liquor and 7 parts by weight of pulverulent silica gel, and the mixture is ground in a hammer mill (comprises 80% by weight of active compound).

VI. 90 parts by weight of a compound according to the invention are mixed with 10 parts by weight of N-methyl-α-pyrrolidone, which gives a solution which is suitable for use in the form of microdrops (comprises 90% by weight of active compound).

VII. 20 parts by weight of a compound according to the invention are dissolved in a mixture composed of 40 parts by weight of cyclohexanone, 30 parts by weight of isobutanol, 20 parts by weight of the adduct of 7 mol of ethylene oxide and 1 mol of isooctylphenol and 10 parts by weight of the adduct of 40 mol of ethylene oxide and 1 mol of castor oil. Pouring the solution into 100,000 parts by weight of water and finely distributing it therein gives an aqueous dispersion which comprises 0.02% by weight of the active compound.

VIII. 20 parts by weight of a compound according to the invention are mixed thoroughly with 3 parts by weight of sodium diisobutylnaphthalene-α-sulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

The active compounds can be used as such, in the form of their formulations or the use forms prepared therefrom, e.g. in the form of directly sprayable solutions, powders, suspensions or dispersions, emulsions, oil dispersions, pastes, dusts, materials for scattering, or granules, by means of spraying, atomizing, dusting, scattering or pouring. The use forms depend entirely on the intended purposes; in any case, they should ensure the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances as such or dissolved in an oil or solvent can be homogenized in water by means of wetting agent, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use products can be varied within substantial ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even the active compound without additives.

Various types of oils, herbicides, fungicides, other pesticides, or bactericides may be added to the active compounds, if appropriate also only immediately prior to use (tank mix). These agents can be admixed with the agents according to the invention in a weight ratio of 1:10 to 10:1.

In the use form as fungicides, the compositions according to the invention can also be present together with other active compounds, e.g. with herbicides, insecticides, growth regulators, fungicides or else with fertilizers. Mixing the compounds I or the compositions comprising them in the use form as fungicides with other fungicides frequently results in a broader fungicidal spectrum of action.

The following list of fungicides together with which the compounds according to the invention can be used is intended to illustrate the possible combinations, but not to impose any limitation:

sulfur, dithiocarbamates and their derivatives, such as iron (III) dimethyldithiocarbamate, zinc dimethyldithiocarbamate, zinc ethylenebisdithiocarbamate, manganese ethylenebisdithiocarbamate, manganese zinc ethylenediaminebisdithiocarbamate, tetramethylthiuram disulfides, ammonia complex of zinc (N,N-ethylenebisdithiocarbamate), ammonia complex of zinc (N,N′-propylenebisdithiocarbamate), zinc (N,N′-propylenebisdithiocarbamate), N,N′-polypropylenebis(thiocarbamoyl)disulfide;

nitro derivatives, such as dinitro-(1-methylheptyl)phenyl crotonate, 2-sec-butyl-4,6-dinitrophenyl 3,3-dimethylacrylate, 2-sec-butyl-4,6-dinitrophenylisopropyl carbonate, diisopropyl 5-nitro-isophthalate;

heterocyclic substances, such as 2-heptadecyl-2-imidazoline acetate, 2,4-dichloro-6-(o-chloroanilino)-s-triazine, O,O-diethyl phthalimidophosphonothioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-methoxycarbonylaminobenzimidazole, 2-(2-furyl)benzimidazole, 2-(4-thiazolyl)benzimidazole, N-(1,1,2,2-tetrachloroethylthio)tetrahydrophthalimide, N-trichloromethylthiotetrahydrophthalimide, N-trichloromethylthiophthalimide,

N-dichlorofluoromethylthio-N′,N′-dimethyl-N-phenylsulfodiamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, pyridine-2-thiol 1-oxide, 8-hydroxyquinoline or its copper salt, 2,3-dihydro-5-carboxanilido-6-methyl-1,4-oxathiine, 2,3-dihydro-5-carboxanilido-6-methyl-1,4-oxathiine 4,4-dioxide, 2-methyl-5,6-dihydro-4H-pyran-3-carboxanilide, 2-methylfuran-3-carboxanilide, 2,5-dimethylfuran-3-carboxanilide, 2,4,5-trimethylfuran-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine-2,2,2-trichloroethyl acetal, piperazine-1,4-diylbis-1-(2,2,2-trichloroethyl)formamide, 1-(3,4-dichloroanilino)-1-formylamino-2,2,2-trichloroethane; 2,6-dimethyl-N-tridecylmorpholine or its salts, 2,6-dimethyl-N-cyclododecylmorpholine or its salts, N-[3-(p-tert-butylphenyl)-2-methylpropyl]-cis-2,6-dimethyl- morpholine, N-[3-(p-tert-butylphenyl)-2-methylpropyl]piperidine,

1-[2-(2,4-dichlorophenyl)-4-ethyl-1,3-dioxolan-2-ylethyl]-1H-1,2,4-triazole, 1-[2-(2,4-dichlorophenyl)-4-n-propyl-1,3-dioxolan-2-ylethyl]-1H-1,2,4-triazole, N-(n-propyl)-N-(2,4,6-trichlorophenoxyethyl)-N′-imidazolylurea, 1-(4-chlorophenoxy)-3,3-dimethyl-1-(lH-1,2,4-triazol-1-yl)-2-butanone, 1-(4-chlorophenoxy)-3,3-dimethyl-1-(lH-1,2,4-triazol-1-yl)-2-butanol, (2RS,3RS)-1-[3-(2-chlorophenyl)-2-(4-fluorophenyl)oxiran-2-ylmethyl]-1H-1,2,4-triazole, α-(2-chlorophenyl)-α-(4-chlorophenyl)-5-pyrimidinemethanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene, 1,2-bis(3-methoxycarbonyl-2-thioureido)benzene,

strobilurines such as methyl E-methoxyimino-[α-(o-tolyloxy)-o-tolyl]acetate, methyl E-2-{2-[6-(2-cyanophenoxy)pyrimidin-4-yloxy]phenyl}-3-methoxyacrylate, methyl-E-methoxyimino-[α-(2-phenoxyphenyl)]acetamide, methyl E-methoxyimino-[α-(2,5-dimethylphenoxy)-o-tolyl]acetamide, methyl E-2-{2-[(2-trifluoromethylpyrid-6-yl)oxymethyl]phenyl}-3-methoxyacrylate, methyl (E,E)-methoximino-(2-[1-(3-trifluoromethylphenyl) ethylideneaminooxymethyl]phenyl}acetate, methyl N-(2-{[1-(4-chlorphenyl)-1H-pyrazol-3-yl]oxymethyl}phenyl)-N-methoxycarbamate,

anilinopyrimidines such as N-(4,6-dimethylpyrimidin-2-yl)aniline, N-[4-methyl-6-(1-propynyl)pyrimidin-2-yl]aniline, N-[4-methyl-6-cyclopropylpyrimidin-2-yl]aniline,

phenylpyrroles such as 4-(2,2-difluoro-1,3-benzodioxol-4-yl)pyrrole-3-carbonitrile,

cinnamamides such as 3-(4-chlorophenyl)-3-(3,4-dimethoxyphenyl)acryloylmorpholine,

and a variety of fungicides such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, DL-N-(2,6-dimethylphenyl)-N-(2′-methoxyacetyl)alanine methyl ester, N-(2,6-dimethylphenyl)-N-chloroacetyl-D,L-2-amino-butyrolactone, DL-N-(2,6-dimethylphenyl)-N-(phenylacetyl)alanine methyl ester, 5-methyl-5-vinyl-3-(3,5-dichlorophenyl)-2,4-dioxo-1,3-oxazolidine, 3-(3,5-dichlorophenyl)5-methyl-5-methoxymethyl-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-[N-(ethylaminocarbonyl)-2-methoximino]acetamide, 1-[2-(2,4-dichlorophenyl)pentyl]-1H-1,2,4-triazole, 2,4-difluoro-a-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### SYNTHESIS EXAMPLES

With due modification of the starting materials, the protocols shown in the synthesis examples below were used for obtaining further compounds I. The resulting compounds, together with physical data, are listed in the tables which follow.

### Example 1

### Preparation of 3-butylamino-2-phenylimidazo(1,2-a)pyridine [I-1]

0.47 g (5 mmol) of 2-aminopyridine and 0.636 g (6 mmol) of benzaldehyde were dissolved in 10 ml of a methanol/CH₂Cl₂ mixture (3:1), and 0.12 g (0.25 mmol) of scandium(III) trifluoromethanesulfonate was added. The resulting suspension was stirred at 20-25° C. for 30 min and then admixed with 0.5 g (6 mmol) of n-butyl isocyanide. The mixture was stirred at 20-25° C. for 15 hours, and 7 g of a freshly conditioned acidic ion exchanger were then added. The mixture was stirred for 10 min and then filtered off, and the ion exchanger was washed. The filtrate was discarded. The ion exchanger was eluted with 2% NH₃ solution and the resulting filtrate was evaporated under reduced pressure. This gave 1.2 g of a 90% pure product.

IR: 2956, 2929, 1502, 1444, 1357, 1345, 751, 697 cm⁻¹

TABLE I

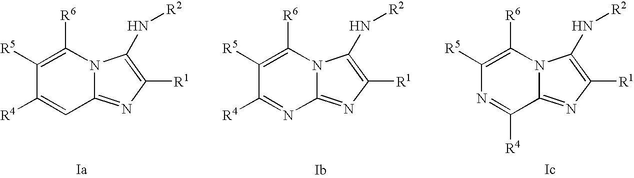

| | | | | | | | Phys. data (m.p. [° C.], |
|---|---|---|---|---|---|---|---|
| No. | Formula | R¹ | R² | R⁴ | R⁵ | R⁶ | IR [cm⁻¹], ¹H-NMR [δ {ppm}]) |
| I-1 | Ia | C₆H₅ | (CH₂)₃CH₃ | H | H | H | see Ex. 1 |
| I-2 | Ia | 2,6-F₂—C₆H₃ | (CH₂)₃CH₃ | H | H | H | 2958, 1464, 999, 788, 737 |
| I-3 | Ia | 2-F—C₆H₄ | (CH₂)₃CH₃ | H | H | H | 2957, 1566, 1451, 752 |
| I-4 | Ia | 3-F—C₆H₄ | (CH₂)₃CH₃ | H | H | H | 1614, 1584, 1203, 756, 683 |
| I-5 | Ia | 4-F—C₆H₄ | (CH₂)₃CH₃ | H | H | H | 2930, 1507, 1221, 841, 752 |
| I-6 | Ia | 2-OCF₂CF₂H—C₆H₄ | (CH₂)₃CH₃ | H | H | H | 1191, 1119, 750 |
| I-7 | Ia | 2-Cl,6-F—C₆H₃ | (CH₂)₃CH₃ | H | H | H | 2957, 2871, 1446, 894, 740 |
| I-8 | Ib | 2,6-F₂—C₆H₃ | (CH₂)₃CH₃ | H | H | H | 2958, 1465, 1562, 1000, 788 |
| I-9 | Ib | 2-F—C₆H₄ | CH(CH₃)₂ | H | H | H | oil |
| I-10 | Ia | 2-F—C₆H₄ | CH(CH₃)₂ | H | H | H | 2958, 1569, 1511, 1491, 1441, |
| | | | | | | | 1237, 916, 789, 765, 702 |
| I-11 | Ib | 2-Cl,6-F—C₆H₃ | (CH₂)₃CH₃ | H | H | H | oil |
| I-12 | Ib | C₆H₅ | (CH₂)₃CH₃ | H | H | H | 1611, 1213, 767, 704 |
| I-13 | Ib | 2-F—C₆H₄ | (CH₂)₃CH₃ | H | H | H | 2957, 1613, 760 |
| I-14 | Ia | 2-Cl—C₆H₄ | (CH₂)₃CH₃ | H | H | H | 2956, 1351, 751 |
| I-15 | Ia | 2,6-Cl₂—C₆H₃ | (CH₂)₃CH₃ | H | H | H | 2957, 1458, 786, 740 |
| I-16 | Ia | 2,6-F₂—C₆H₃ | cyclo-C₆H₁₁ | H | H | H | 2929, 1631, 1464, 999, 737 |
| I-17 | Ia | C₆H₅ | cyclo-C₆H₁₁ | H | H | H | 181-183 |
| I-18 | Ib | 3-F—C₆H₄ | (CH₂)₃CH₃ | H | H | H | 2957, 1613, 1178, 786 |
| I-19 | Ib | 2-OCF₂CF₂H—C₆H₄ | (CH₂)₃CH₃ | H | H | H | oil |
| I-20 | Ia | C₆F₅ | (CH₂)₃CH₃ | H | H | H | 1630, 1491, 989, 766 |
| I-21 | Ib | 4-F—C₆H₄ | (CH₂)₃CH₃ | H | H | H | 2958, 1504, 1220, 842 |
| I-22 | Ib | 2,6-Cl₂—C₆H₃ | (CH₂)₃CH₃ | H | H | H | oil |
| I-23 | Ic | 2,6-F₂—C₆H₃ | (CH₂)₃CH₃ | H | H | H | 9.0 (s); 8.1 (s); 7.8 (m); 7.4 |
| | | | | | | | (m); 7.1 (m); 3.0 (q); 1.4 (m); |
| | | | | | | | 1.3 (m); 0.8 (t) |
| I-24 | Ic | 4-F—C₆H₄ | (CH₂)₃CH₃ | H | H | H | oil |
| I-25 | Ic | 3-F—C₆H₄ | (CH₂)₃CH₃ | H | H | H | oil |
| I-26 | Ic | 2-OCF₂CF₂H—C₆H₄ | (CH₂)₃CH₃ | H | H | H | 9.0 (s); 8.0 (s); 7.8 (m); |
| | | | | | | | 7.75 (m); 7.5 (m); 5.8 (t); 2.9 |
| | | | | | | | (q); 1.3 (m); 1.3 (m) 0.8 (t) |
| I-27 | Ic | 2-Cl,6-F—C₆H₃ | (CH₂)₃CH₃ | H | H | H | 9.0 (s); 8.0 (s); 7.8 (m); 7.3 |
| | | | | | | | (m); 7.2 (m); 3.0 (m); 1.3 (m); |
| | | | | | | | 1.2 (m); 0.8 (t) |
| I-28 | Ic | 2-Cl—C₆H₄ | (CH₂)₃CH₃ | H | H | H | 9.0 (s); 8.0 (s); 7.8 (m); 7.6 |
| | | | | | | | (m); 7.5 (m); 7.4 (m); 3.0 (m); |
| | | | | | | | 1.3 (m); 1.2 (m); 0.8 (t) |
| I-29 | Ic | 3-Cl—C₆H₄ | (CH₂)₃CH₃ | H | H | H | oil |
| I-30 | Ic | 2,6-Cl₂—C₆H₃ | (CH₂)₃CH₃ | H | H | H | 9.0 (s); 8.0 (s); 7.8 (m); 7.5 |
| | | | | | | | (m); 7.2 (m); 3.0 (m); 1.3 (m); |
| | | | | | | | 1.2 (m); 0.8 (t) |
| I-31 | Ic | 2-CH₃—C₆H₄ | (CH₂)₃CH₃ | H | H | H | 9.0 (s); 7.95 (m); 7.83 (m); 7.3 |
| | | | | | | | (m); 7.25 (m); 3.0 (q); 1.4 (m); |
| | | | | | | | 1.3 (m); 0.8 (t) |
| I-32 | Ic | 3-CH₃—C₆H₄ | (CH₂)₃CH₃ | H | H | H | oil |
| I-33 | Ic | 4-CH₃—C₆H₄ | (CH₂)₃CH₃ | H | H | H | 94-97 |
| I-34 | Ic | 2,4-(CH₃)₂—C₆H₃ | (CH₂)₃CH₃ | H | H | H | oil |
| I-35 | Ic | 2-(OCH₂—C₆H_{5)—C}₆H₄ | (CH₂)₃CH₃ | H | H | H | 9.0 (s); 7.9 (s); 7.8 (m); |
| | | | | | | | 7.3-7.1 (m); 5.10 (s); 2.8 (q); |
| | | | | | | | 1.3 (m); 0.8 (t) |
| I-36 | Ic | 4-OCH₃—C₆H₄ | (CH₂)₃CH₃ | H | H | H | oil |
| I-37 | Ic | 2-OCH₃—C₆H₄ | (CH₂)₃CH₃ | H | H | H | oil |
| I-38 | Ic | 2,4,6-(OCH₃)₃—C₆H₂ | (CH₂)₃CH₃ | H | H | H | 135-136 |
| I-39 | Ic | 3,4,5-(OCH₃)₃—C₆H₂ | (CH₂)₃CH₃ | H | H | H | 118-121 |
| I-40 | Ic | 2,3,4-(OCH₃)₃—C₆H₂ | (CH₂)₃CH₃ | H | H | H | 9.0 (s); 8.0 (s); 7.9 (m); 7.5 |
| | | | | | | | (d); 6.9 (d); 4.0 (s); 3.9 (s); |
| | | | | | | | 2.9 (q); 1.3 (m); 0.8 (t) |
| I-41 | Ic | C₆H₅ | (CH₂)₃CH₃ | H | H | H | oil |
| I-42 | Ia | 2,6-F₂—C₆H₃ | C(CH₃)₃ | H | H | H | 147-152 |
| I-43 | Ia | 2-Cl,6-F—C₆H₃ | C(CH₃)₃ | H | H | H | 153-156 |
| I-44 | Ic | 2,6-F₂—C₆H₃ | C(CH₃)₃ | H | H | H | 150-154 |
| I-45 | Ic | 2-Cl,6-F—C₆H₃ | C(CH₃)₃ | H | H | H | 165-170 |
| I-46 | Ic | 2,6-F₂—C₆H₃ | cyclo-C₆H₁₁ | H | H | H | 130-135 |
| I-47 | Ic | 2-Cl,6-F—C₆H₃ | cyclo-C₆H₁₁ | H | H | H | 116-119 |
| I-48 | Ia | 2,4,6-F₃—C₆H₂ | C(CH₃)₃ | H | H | H | 122-124 |
| I-49 | Ic | 2,4,6-F₃—C₆H₂ | C(CH₃)₃ | H | H | H | 120-125 |
| I-50 | Ia | 2,4,6-F₃—C₆H₂ | cyclo-C₆H₁₁ | H | H | H | 107-108 |
| I-51 | Ic | 2,4,6-F₃—C₆H₂ | cyclo-C₆H₁₁ | H | H | H | 124-128 |
| I-52 | Ia | 2,6-F₂—C₆H₃ | (CH₃)₃SiCH₂ | H | H | H | 2953, 1464, 1248, 999, 861 |
| I-53 | Ia | 2,4,6-F₃—C₆H₂ | (CH₃)₃SiCH₂ | H | H | H | 97-100 |
| I-54 | Ia | quinolin-7-yl | (CH₂)₃CH₃ | H | H | H | 150-154 |
| I-55 | Ia | 2-naphthyl | (CH₂)₃CH₃ | H | H | H | 107-110 |
| I-56 | Ia | 2-NO₂-thiophen-4-yl | (CH₂)₃CH₃ | H | H | H | 168-170 |
| I-57 | Ia | 2-(SCH₃)-4,6-(OCH₃)₂₋ | (CH₂)₃CH₃ | H | H | H | 108-113 |
| | | pyrimidin-5-yl | | | | | |
| I-58 | Ia | 1-(CH(CH₃)₂- | (CH₂)₃CH₃ | H | H | H | 90-94 |
| | | pyrazol-4-yl | | | | | |
| I-59 | Ia | thiophen-3-yl | (CH₂)₃CH₃ | H | H | H | 92-97 |
| I-60 | Ia | 2, 5-Br₂- | (CH₂)₃CH₃ | H | H | H | 2995, 1442, 1353, 966, 752 |
| | | thiophen-3-yl | | | | | |
| I-61 | Ia | 4,5-Cl₂- | (CH₂)₃CH₃ | H | H | H | 2956, 1500, 1352, 1316, 750 |
| | | thiophen-3-yl | | | | | |
| I-62 | Ia | 3-(OC₆H₅)—C₆H₄ | (CH₂)₃CH₃ | H | H | H | 2956, 2929, 1581, 1503, 1489, |
| | | | | | | | 1455, 1444, 1346, 1268, 1239, |
| | | | | | | | 1223, 903, 752, 737, 691 |
| I-63 | Ic | 1-[CH(CH₃)₂]- | (CH₂)₃CH₃ | H | H | H | 95-98 |
| | | pyrazol-4-yl | | | | | |
| I-64 | Ic | thiophen-3-yl | (CH₂)₃CH₃ | H | H | H | 106-109 |
| I-65 | Ia | 4-pyridyl | (CH₂)₃CH₃ | H | H | H | 95-98 |
| I-66 | Ia | 3-pyridyl | (CH₂)₃CH₃ | H | H | H | oil |
| I-67 | Ia | furan-3-yl | (CH₂)₃CH₃ | H | H | H | 75-78 |
| I-68 | Ic | 2,5-Br₂-thio- | (CH₂)₃CH₃ | H | H | H | 88-92 |
| | | phen-3-yl | | | | | |
| I-69 | Ic | 2-(SCH₃)-4,6-(OCH₃)₂- | (CH₂)₃CH₃ | H | H | H | 111-114 |
| | | pyrimidin-5-yl | | | | | |
| I-70 | Ic | furan-3-yl | (CH₂)₃CH₃ | H | H | H | 101-104 |
| I-71 | Ic | 3-pyridyl | (CH₂)₃CH₃ | H | H | H | 2957, 1547, 1351, 710 |
| I-72 | Ic | 1-[C(CH₃)₃]- | (CH₂)₃CH₃ | H | H | H | 2930, 1589, 1292, 788 |
| | | pyrrol-3-yl | | | | | |
| I-73 | Ic | 4,5-Cl₂- | (CH₂)₃CH₃ | H | H | H | 2957, 1576, 1532, 1351, 907 |
| | | thiophen-3-yl | | | | | |
| I-74 | Ic | 2-naphthyl | (CH₂)₃CH₃ | H | H | H | 2956, 1533, 1356, 823, 752 |
| I-75 | Ic | 1-(4-Cl—C₆H₄)-3,5- | (CH₂)₃CH₃ | H | H | H | 94-96 |
| | | (CH₃)₂-pyrazol-4-yl | | | | | |
| I-76 | Ic | quinolin-7-yl | (CH₂)₃CH₃ | H | H | H | 2957, 2870, 1548, 1494, 1352, 843 |
| I-77 | Ic | 1-[C(CH₃)₃]- | (CH₂)₃CH₃ | H | H | H | oil |
| | | pyrazol-4-yl | | | | | |
| I-78 | Ic | 3-CF₃—C₆H₄ | (CH₂)₃CH₃ | H | H | H | oil |
| I-79 | Ic | 2-CF₃—C₆H₄ | (CH₂)₃CH₃ | H | H | H | 89-92 |
| I-80 | Ic | 4-OCF₂CF₂H—C₆H₄ | (CH₂)₃CH₃ | H | H | H | 87-90 |
| I-81 | Ic | 2,3-Cl₂—C₆H₃ | (CH₂)₃CH₃ | H | H | H | oil |
| I-82 | Ic | 3,5-Cl₂—C₆H₃ | (CH₂)₃CH₃ | H | H | H | 121-124 |
| I-83 | Ic | 2,5-Cl₂—C₆H₃ | (CH₂)₃CH₃ | H | H | H | oil |
| I-84 | Ic | 2-Br-4-F—C₆H₃ | (CH₂)₃CH₃ | H | H | H | oil |
| I-85 | Ic | 2-Br—C₆H₄ | (CH₂)₃CH₃ | H | H | H | oil |
| I-86 | Ic | 4-N(CH₃)₂—C₆H₄ | (CH₂)₃CH₃ | H | H | H | 152-154 |
| I-87 | Ic | 2,4,6-F₃—C₆H₂ | (CH₂)₃CH₃ | H | H | H | 98-100 |
| I-88 | Ic | 4-Cl—C₆H₄ | (CH₂)₃CH₃ | H | H | H | 110-112 |
| I-89 | Ic | 2,5-F₂—C₆H₃ | (CH₂)₃CH₃ | H | H | H | oil |
| I-90 | Ic | 1,2,5-(CH₃)₃- | (CH₂)₃CH₃ | H | H | H | 2929, 1593, 1549, 1296 |
| | | pyrrol-3-yl | | | | | |
| I-91 | Ic | 2,5-Br₂- | CH(CH₃)₂ | H | H | H | 2965, 1440, 1351, 1001 |
| | | thiophen-3-yl | | | | | |
| I-92 | Ic | 4,5-Cl₂- | CH(CH₃)₂ | H | H | H | 120-122 |
| | | thiophen-3-yl | | | | | |
| I-93 | Ic | isoquinolin-7-yl | CH(CH₃)₂ | H | H | H | 142-144 |
| I-94 | Ic | 2-naphthyl | CH(CH₃)₂ | H | H | H | 136-138 |
| I-95 | Ib | 2,5-Br₂- | CH(CH₃)₂ | H | H | H | 2965, 1613, 1285, 998, 766 |
| | | thiophen-3-yl | | | | | |
| I-96 | Ib | thiophen-3-yl | CH(CH₃)₂ | H | H | H | 132-135 |
| I-97 | Ib | 2-naphthyl | CH(CH₃)₂ | H | H | H | 181-183 |
| I-98 | Ib | 1-[C(CH₃)₃]- | CH(CH₃)₂ | H | H | H | 2971, 1612, 1267, 763 |
| | pyrrol-3-yl | | | | | | |
| I-99 | Ib | 1-[C(CH₃)₃]- | CH(CH₃)₂ | H | H | H | 162-165 |
| | | pyrazol-4-yl | | | | | |
| I-100 | Ib | 2-(SCH₃)-4,6-(OCH₃)₂- | CH(CH₃)₂ | H | H | H | 169-173 |
| | | pyrimidin-5-yl | | | | | |
| I-101 | Ib | furan-3-yl | CH(CH₃)₂ | H | H | H | 115-117 |
| I-102 | Ib | 3-(OC₆H₅)—C₆H₄ | CH(CH₃)₂ | H | H | H | 110-113 |
| I-103 | Ic | 3-(OC₆H₅)—C₆H₄ | (CH₂)₃CH₃ | H | H | H | 2957, 2929, 1608, 1591, 1579, |
| | | | | | | | 1548, 1533, 1456, 1441, 1426, |
| | | | | | | | 1351, 1238, 1217, 691 |
| I-104 | Ic | 2-NO₂-thiophen-4-yl | (CH₂)₃CH₃ | H | H | H | 107-111 |
| I-105 | Ib | 4,5-Cl₂- | CH(CH₃)₂ | H | H | H | 218-220 |
| | | thiophen-3-yl | | | | | |
| I-106 | Ib | 1-(4-Cl—C₆H₄)-3,5- | CH(CH₃)₂ | H | H | H | 175-177 |
| | | (CH₃)₂-pyrazol-4-yl | | | | | |
| I-107 | Ib | isoquinolin-7-yl | CH(CH₃)₂ | H | H | H | 169-170 |
| I-108 | Ia | 3-F—C₆H₄ | CH(CH₃)₂ | H | H | H | 125-127 |
| I-109 | Ia | C₆H₅ | CH(CH₃)₂ | H | H | H | 139-143 |
| I-110 | Ia | C₆H₅ | CH(CH₃)C(CH₃)₃ | H | H | H | 122-124 |
| I-111 | Ia | 2,4,6-F₃—C₆H₂ | CH(CH₃)C(CH₃)₃ | H | H | H | 2966, 1643, 1599, 1505, 1497, |
| | | | | | | | 1439, 1395, 1375, 1353, 1122, |
| | | | | | | | 1033, 999, 840, 753, 745 |
| I-112 | Ia | 3-(OCH₂C₆H₅)C₆H₄ | CH(CH₃)C(CH₃)₃ | H | H | H | 153-156 |
| I-113 | Ia | 2-Cl,6-F—C₆H₃ | (CH₂)₃CH₃ | CH₃ | H | H | 2957, 2930, 1644, 1567, 1503, |
| | | | | | | | 1466, 1445, 1380, 1353, 1248, |
| | | | | | | | 1197 |
| I-114 | Ia | 2-Cl,6-F—C₆H₃ | (CH₂)₃CH₃ | H | CH₃ | H | 69-70 |
| I-115 | Ia | 2-Cl,6-F—C₆H₃ | (CH₂)₃CH₃ | H | H | H | 2957, 2930, 1613, 1568, 1512, |
| | | | | | | | 1466, 1446, 1391, 1317, 1248, |
| | | | | | | | 897, 777 |
| I-116 | Ia | 3-Cl—C₆H₄ | (CH₂)₃CH₃ | H | CF₃ | H | 74-75 |
| I-117 | Ic | 2,4,6-F₃—C₆H₂ | CH(CH₃)C(CH₃)₃ | H | H | H | 149-152 |
| I-118 | Ic | 3-CH₃—C₆H₄ | CH(CH₃)C(CH₃)₃ | H | H | H | 121-123 |
| I-119 | Ic | cyclo-C₈H₁₅ | CH(CH₃)₂ | H | H | H | 3253, 2962, 2852, 1535, 1491, |
| | | | | | | | 1465, 1445, 1423, 1381, 1348, |
| | | | | | | | 1284, 1173, 1149, 793 |
| I-120 | Ib | 4-CH₃-cyclo-C₆H₁₀ | CH(CH₃)₂ | H | H | H | 163-167 |
| I-121 | Ib | 3-CH₂CH₂CH₃- | CH(CH₃)₂ | H | H | H | 135-137 |
| | | cyclo-C₅H₈ | | | | | |

### Examples of the Activity Against Harmful Fungi

The fungicidal action of the compounds of the formula I was demonstrated by the following experiments:

The active compounds were formulated, separately or jointly, as a 10% emulsion in a mixture of 70% by weight of cyclohexanone, 20% by weight of Nekanil® LN (Lutensol® AP6, wetting agent having emulsifying and dispersant action based on ethoxylated alkylphenols) and 10% by weight of Wettol® EM (nonionic emulsifier based on ethoxylated castor oil) and diluted with water to give the desired concentration.

### Example 1

### Activity Against Alternaria solani on Tomatoes

Leaves of potted plants of the cultivar “Groβe Fleischtomate St. Pierre” were sprayed to run-off with an aqueous suspension made with a stock solution of 10% of active compound, 63% of cyclohexanone and 27% of emulsifier. The next day, the leaves were infected with an aqueous zoospore suspension of Alternaria solani in a 2% biomalt solution having a density of 0.17×10⁶ spores/ml. The plants were subsequently placed in a water vapor-saturated chamber at from 20 to 22° C. After 5 days, the foliar blight had developed on the untreated, but infected, control plants to such an extent that it was possible to evaluate the disease level visually in percent.

In this test, the plants which had been treated with 250 ppm of the compounds I-13 to I-16, I-51, I-66, I-67, I-78 and I-112 showed an infection of not more than 20%, whereas the untreated plants were 90% infected.

### Example 2

### Activity Against Plasmopara viticola

Leaves of potted vines of the cultivar “Müller-Thurgau” were sprayed to run-off with an aqueous preparation of active compound made with a stock solution of 10% of active compound, 63% of cyclohexanone and 27% of emulsifier. The next day, the undersides of the leaves were inoculated with an aqueous zoospore suspension of Plasmopara viticola. The grapevines were subsequently placed initially in a water vapor-saturated chamber at 24° C. for 48 hours and then in a greenhouse at from 20 to 30° C. for 5 days. After this time, the plants were once more placed in a humid chamber for 16 hours to promote the eruption of sporangiophores. The extent of the fungal infection on the underside of the leaves was then determined visually.

In this test, the plants which had been treated with 250 ppm of the compounds I-3, I-5, I-6, I-11, I-12, I-14 to I-16, I-21 to I-23, I-29, I-31 to I-41, I-44, I-49, I-54, I-57, I-61, I-66 to I-69, I-72 to I-81, I-85, I-86, I-88, I-92, I-94, I-101 to 104, I-107, I-110, I-116, I-118, I-120 and I-121 showed an infection of not more than 20%, whereas the untreated plants were 85% infected.

We claim:

1. The use of substituted imidazoazines of the formula I 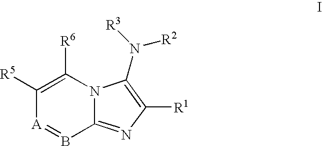
where
R¹ is C₁-C₁₀-alkyl, phenyl, phenyl-C₁-C₄-alkyl, naphthyl, anthracenyl, C₃-C₁₂-cycloalkyl,
where the hydrocarbon radicals are unsubstituted or partially or fully halogenated or may carry one to five identical or different groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₃-C₆-alkenyloxy, C₃-C₆-alkynyloxy or C₁-C₄-alkylenedioxy, which may be halogenated,
phenyl, phenoxy, phenyl-C₁-C₄-alkyl, phenyl-C₁-C₄-alkoxy, or
5- or 6-membered hetaryl or 5- or 6-membered heterocyclyl, containing one to three nitrogen atoms and/or one oxygen or sulfur atom or one or two oxygen and/or sulfur atoms, or
fused 8- to 12-membered hetaryl or fused 8- to 12-membered heterocyclyl, containing one to four nitrogen atoms and/or one oxygen or sulfur atom or one or two oxygen and/or sulfur atoms,
where the ring systems are unsubstituted or substituted by one to three radicals R^{b}:
R^{b} is cyano, nitro, amino, aminocarbonyl, aminothiocarbonyl, halogen, hydroxyl, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyloxy, C₂-C₆-alkynyloxy, phenyl, phenoxy, benzyl, benzyloxy, 5- or 6-membered heterocyclyl, 5- or 6-membered hetaryl or 5- or 6-membered hetaryloxy, containing one to three nitrogen atoms and/or one oxygen or sulfur atom or one or two oxygen and/or sulfur atoms,
where the cyclic groups R^{a} and R^{b} for their part are unsubstituted or substituted by one to three groups R^{c}:
R^{c} is cyano, nitro, halogen, hydroxyl, amino, aminocarbonyl, aminothiocarbonyl, C₁-C₆-alkyl, C₁-C₆-haloalkyl, C₁-C₆-alkylsulfonyl, C₁-C₆-alkylsulfoxyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heterocyclyl,
5- or 6-membered hetaryl, 5- or 6-membered hetaryloxy, where the heterocyclic rings are defined as under R^{b};
R², R³ independently of one another are hydrogen, C₁-C_{C}₁₀-alkyl, C₃-C₁₀-alkenyl, C₃-C₁₀-alkynyl, C₁-C_{C}₁₀-haloalkyl, C₃-C₁₀-haloalkenyl, C₃-C₁₀-haloalkynyl, tri(C₁-C₄-alkyl)silyl-C₁-C₄-alkyl, phenyl, phenyl-C₁-C₄-alkyl, or
R², R³ together with the bridging nitrogen atom form a 5- or 6-membered heterocyclic or heteroaromatic ring which may be interrupted by one to three nitrogen atoms and/or one oxygen or sulfur atom or one or two oxygen and/or sulfur atoms and which may be substituted by one or two groups R^{b};
where the cyclic radicals R² and/or R³ may be substituted by one to four groups R^{c};
A, B independently of one another are N or CR⁴;
R⁴, R⁵, R⁶ independently of one another are hydrogen, halogen, cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, amino, C₁-C₆-alkylamino, di-C₁-C₆-alkylamino, C₂-C₆-alkenyl, C₃-C₆-alkenyloxy, C₂-C₆-alkynyl or C₃-C₆-alkynyloxy
for controlling phytopathogenic harmful fungi.

2. A compound of the formula IA 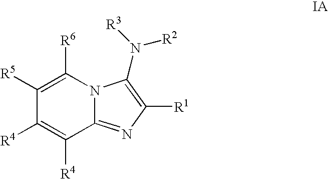
where the variables are as defined in claim 1, except that the group —NR²R³ may not have the meanings amino, methylamino and dimethylamino, with the proviso that compounds having substituents of the following combinations are excluded:
(A) R¹ C₁-C₃-alkyl, unsubstituted or substituted by hydroxyl or phenyl, phenylethylene, cyclohexyl, phenyl which may be substituted by methoxy, methyl, benzyloxy or methoxycarbonyl,
R² tert-butyl, octyl, phenylethylene, phenyl, unsubstituted or substituted by methyl, methoxy or thiomethyl, benzyl, unsubstituted or substituted by methoxy or halogen,
R³-R⁶ hydrogen;
and
(B) R¹ cyclohexyl or phenyl which may be substituted by nitro or methoxy;
R² tert-butyl;
R³, R⁶ hydrogen; and either
R⁵ methyl or halogen and R⁴ hydrogen; or
R⁴ and R⁵ meta-dibromo or meta-dihalogen.

3. A compound of the formula IB 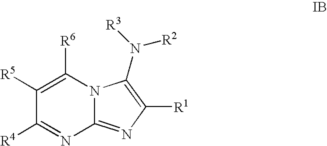
where the variables are as defined in claim 1, except that the group —NR²R³ may not have the meanings amino, methylamino and dimethylamino, with the proviso that compounds having substituents of the following combinations are excluded:
R¹ phenyl which may be substituted by methoxy or methoxycarbonyl;
R² octyl, benzyl or phenylethylene, where the phenyl groups may be substituted by halogen, methoxy or
R³-R⁶ hydrogen.

4. A compound of the formula IC 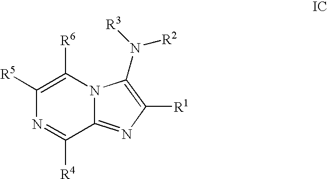
where the variables are as defined in claim 1, except that the group —NR²R³ may not have the meanings amino, methylamino and dimethylamino, with the proviso that compounds having substituents of the following combinations are excluded:
R¹ C₁-C₃-alkyl, unsubstituted or substituted by hydroxyl, phenyl which may be substituted by methoxy, benzyloxy or methoxycarbonyl;
R² benzyl or octyl;
R³-R⁶ hydrogen.

5. A compound of the formulae IA, IB and IC as claimed in any of claims 2 to 4 in which R¹ is substituted phenyl.

6. A compound of the formulae IA, IB and IC as claimed in any of claims 2 to 5 in which R⁴ is hydrogen.

7. A compound of the formulae IA, IB and IC as claimed in any of claims 2 to 6 in which R³ is hydrogen.

8. A compound of the formulae IA, IB and IC as claimed in any of claims 2 to 7 in which R⁵ and R⁶ are hydrogen.

9. A process for preparing compounds of the formulae IA, IB and IC as claimed in claim 7, which comprises reacting amino compounds of the formula II, 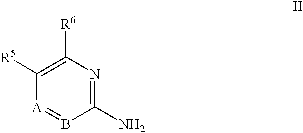
in which the variables are as defined for formula I, with aldehydes of the formula III, 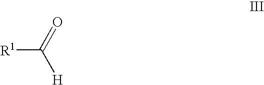
in which R¹ is as defined in formula I, and isonitriles of the formula IV, 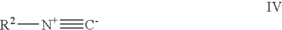
in which R² is as defined in formula I, in the presence of acids or Lewis acids.

10. An intermediate of the formula IV as claimed in claim 9 in which
R² is (R) CH(CH₃)CF₃, (S) CH(CH₃)CF₃, (R) CH(CH₃)C(CH₃)₃ or (S) CH(CH₃)C(CH₃)₃.

11. A composition suitable for controlling phytopathogenic harmful fungi, comprising a solid or liquid carrier and a compound of the formula I as claimed in claim 2.

12. A method for controlling phytopathogenic harmful fungi, which comprises treating the fungi or the materials, plants, the soil or the seeds to be protected against fungal infection with an effective amount of a compound of the formula I as claimed in claim 1.
